SEQUENCE LISTING (3 sequences)

SEQ ID NO: 1 - protein, 14 aa, Artificial
  sequence of T140
  misc_feature (3)..(3): X = l-3-(2-naphthyl)alanine
  misc_feature (8)..(8): X = dLys
  misc_feature (12)..(12): X = l-citrulline
RRXCYRKXPYRXCR

SEQ ID NO: 2 - protein, 14 aa, Artificial sequence
  sequence TN14003
  misc_feature (3)..(3): X = l-3-(2-naphthyl)alanine
  misc_feature (6)..(6): X = l-citrulline
  misc_feature (8)..(8): X = dLys
  misc_feature (12)..(12): X = l-citrulline
RRXCYXKXPYRXCR

SEQ ID NO: 3 - protein, 18 aa, Artificial sequence
  Sequence of T22
RRWCYRKCYKGYCYRKCR

(19) United States
(12) Patent Application Publication
(10) Pub. No.: US 2007/0258893 A1
(43) Pub. Date: Nov. 8, 2007

(54) Compositions and methods for imaging expression of cell surface receptors

(76) Inventors: Hyunsuk Shim, Atlanta, GA (US); Jae Min Jeong, Seoul (KR)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/787,366

(22) Filed: Apr. 16, 2007

Related U.S. Application Data
(63) Continuation-in-part of application No. 10/550,525, now abandoned.
(60) Provisional application No. 60/458,217, filed on Mar. 27, 2003.

(51) Int. Cl. A61K 51/00

(52) U.S. Cl. 424/1.69

(57) ABSTRACT

The present disclosure provides imaging agents and methods for imaging surface cell receptors, particularly CXCR4 receptors and biological conditions associated with the expression of CXCR4 receptors, including, but not limited to, cancer and metastasis. In embodiments, the present disclosure provides radiolabeled CXCR4 peptide antagonists detectable in vivo or in vitro by a PET scanner.

### CROSS REFERENCE TO RELATED APPLICATION

This application is a continuation-in-part application of and claims benefit of and priority to U.S. patent application Ser. No. 10/550,525 filed on Sep. 27, 2005, which claims priority to PCT Patent Application Publication No. PCT/US04/09570 filed on Mar. 26, 2004, which claims priority to U.S. Provisional Patent Application No. 60/458,217 filed on Mar. 27, 2003, all of which are incorporated by reference in their entirety herein.

### FIELD OF THE INVENTION(S)

The present disclosure relates to the field of imaging agents, particularly radiolabeled peptide antagonists of CXCR4 chemokine receptors, even more particularly to radiolabeled peptide antagonists capable of imaging conditions including, but not limited to, cancer and metastasis.

### BACKGROUND

Cancer can be a fatal disease, in part, because cancer can spread or metastasize throughout an organism. Metastasis plays a major role in the morbidity and mortality of many cancers, including breast cancer and most head and neck cancers. Breast cancer metastasizes in a stereotypical pattern resulting in lesions found in the lymph node, lung, liver, and bone marrow. Generally, cancer cells lose differentiated properties, proper tissue compartmentalization, cell-cell attachment as well as obtain altered cell substratum attachment, altered cytoskeletal organization, cell locomotion, and the ability to survive at distant sites. Squamous cell carcinoma (SCC), a malignant tumor of epithelial origin, represents more than 90% of all head and neck cancers. While lymph node metastases are more common in SCCHN patients (˜60%), approximately 20 to 25% of patients with SCCHN develop distant metastases, primarily in the lungs, liver, and bone. SCCHN patients without nodal and distant metastases are likely to have a more favorable prognosis than their counterparts.

Metastasis is the result of several sequential steps and represents an organ-selective process. Although a number of mechanisms have been implicated in the metastasis of head and neck cancer, the precise mechanisms determining the directional migration and invasion of tumor cells into specific organs remain elusive. Chemokines are secreted proteins that act in a coordinated fashion with cell-surface proteins, including integrins, to direct the homing of various subsets of hematopoietic cells to specific anatomical sites. An exemplary chemokine implicated in cancer progression includes CXCR4. CXCR4 mediates the migration of cancer cells to the lymph nodes, lungs, liver, and bones. This migration is mediated through the chemotaxis of CXCR4 toward its ligand, stromal cell-derived factor 1 (SDF-1). While the levels of SDF-1 are high at the common destinations of cancer metastasis including the lymph node, lung, liver, and the bone marrow, the expression of CXCR4 is significantly elevated in malignant tumors compared to their normal tissue counterparts. The interactions between SDF-1 and CXCR4 have been shown to direct cells to organ sites that have high levels of SDF-1 expression, suggesting that these interactions play a key role in the chemotaxis and homing of these metastatic cells

Angiogenesis, the formation of new blood vessels from pre-existing vasculature, is a fundamental process occurring during tumor progression and it depends on the balance between pro-angiogenic molecules and anti-angiogenic molecules. Cancer cells spread throughout the body by metastasis. Interactions between vascular cells and the extracellular matrix (ECM) are involved in multiple steps of tumor angiogenesis and metastasis. CXCR4 is implicated in angiogenesis as well as metastasis.

Positron-emission tomography (PET) has become a generally accepted technology for pre-clinical and clinical non-invasive imaging of diseases, especially cancer. Tracers such as 2-deoxy-2-[¹⁸F]fluoro-D-glucose ([¹⁸F]FDG) and other radiopharmaceuticals that have the ability to target specific cellular and molecular processes have contributed to the rapid progress of PET technology.

PET is a diagnostic examination that involves the acquisition of physiologic images based on the detection of radiation from the emission of positrons. In particular, PET is a nuclear medicine medical imaging technique that produces a three dimensional image or map of functional processes in the body. Positrons are tiny particles emitted from a radioactive substance administered to the patient. The subsequent images of the human body developed with this technique are used to evaluate a variety of diseases.

A short-lived radioactive tracer isotope that decays by emitting a positron, is chemically incorporated into a molecule (e.g., a biologically active molecule, a polypeptide, or polynucleotide) and is injected into the living subject (e.g., usually into blood circulation). There is a waiting period while the molecule becomes concentrated in tissues of interest, then the subject is placed in the imaging scanner. The short-lived isotope decays, emitting a positron. After travelling up to a few millimeters the positron annihilates with an electron, producing a pair of annihilation photons (similar to gamma rays) moving in opposite directions. These are detected when they reach a scintillator material in the scanning device, creating a burst of light that is detected by photomultiplier tubes. The technique depends on simultaneous or coincident detection of the pair of photons: photons that do not arrive in pairs (e.g., within a few nanoseconds) are ignored.

Because annihilation photons are always emitted 180° apart, it is possible to localize their source to a straight-line in space. Using statistics collected from tens-of-thousands of coincidence events, a map of their origin in the body can be plotted. The resulting map shows the tissues in which the molecular probe has become concentrated, and can be interpreted by nuclear medicine physician or radiologist in the context of the patient's diagnosis and treatment plan. While PET is used in clinical oncology (medical imaging of tumors and the search for metastases) and in human brain and heart research, current imaging agents either lack specificity for the cancer, are not accurate predictors of metastasis, or are eliminated too quickly or too slowly from the body for optimal imaging.

### SUMMARY

The present disclosure provides compositions and methods for imaging certain biological conditions associated with expression of CXCR4 receptors in vivo and/or in vitro. Particular aspects of the present disclosure provide imaging compositions and methods for the detection, quantification, or identification of cancer cells and/or cancer cell metastases. The diagnostics include, but are not limited to, labeled CXCR4 antagonists, in particular CXCR4 peptide antagonists. In one aspect, the CXCR4 peptide antagonist is not an antibody or antibody fragment. In an embodiment the antagonist is labeled with a radiolabel for PET or SPECT imaging. In an embodiment, the isotope label is a PET isotope.

Exemplary embodiments of an imaging composition of the present disclosure include a CXCR4 peptide antagonist, where the CXCR4 peptide antagonist is not an antibody or fragment thereof, and a radioisotope coupled to the CXCR4 peptide antagonist, where the radioisotope is detectable by a PET scanner. In embodiments, the CXCR4 peptide antagonist is TN14003 or a derivative thereof. In embodiments the CXCR4 peptide antagonist interferes with ligand binding to a CXCR4 receptor or homologue thereof; in particular, the CXCR4 peptide antagonist prevents the CXCR4 receptor from binding the ligand SDF-1. In embodiments of the imaging compositions of the present disclosure, the radioisotope is ¹⁸F. In some embodiments, the ¹⁸F is coupled to the CXCR4 peptide antagonist via a linker comprising hydrazinonicotinic acid (HYNIC). An exemplary embodiment of an imaging composition of the present disclosure includes a ¹⁸F labeled CXCR4 peptide antagonist, where the CXCR4 peptide antagonist is TN14003 or a derivative thereof, and a pharmaceutically acceptable carrier.

Embodiments of methods of imaging of the present disclosure include providing an imaging probe including a CXCR4 peptide antagonist coupled to a radioisotope, where the CXCR4 peptide antagonist is not an antibody or fragment thereof, contacting a specimen to be imaged with a detectably effective amount of the imaging probe, and making a radiographic image. In particular, the methods include imaging expression of CXCR4 receptors, where the expression of CXCR4 receptors is associated with one or more conditions selected from: inflammation, cancer, a tumor, angiogenesis, and metastasis

Additional embodiments of the disclosure include methods of imaging a condition associated with expression of CXCR4 receptors in a host. Such methods include administering to the host a detectably effective amount of a composition including a radiolabeled CXCR4 peptide antagonist, where the CXCR4 peptide antagonist is not an antibody or fragment thereof, and creating a radiographic image of the location and distribution of the a radiolabeled CXCR4 peptide antagonist in the host with an imaging apparatus. The radiolabeled CXCR4 peptide antagonist binds to CXCR4 receptors, and the intensity of uptake of radiolabeled CXCR4 peptide antagonist is related to the expression level of CXCR4 receptors in the host, where the expression level of CXCR4 receptors is associated with one or more disorders.

In another aspect, the present disclosure provides methods of predicting metastasis of a tumor and/or cancer. An exemplary embodiment of a method of predicting metastasis includes contacting a specimen having tumor cells with a detectably effective amount of a composition of ¹⁸F-TN14003 and creating a radiographic image of the location and distribution of the ¹⁸F-TN14003 in the tumor cells with an imaging apparatus. In such methods, the ¹⁸F-TN14003 binds to CXCR4 receptors, and the intensity of uptake of ¹⁸F-TN14003 by the tumor cells is related to the metastatic potential of the tumor cells.

Aspects of the present disclosure also include methods of determining an effect of a drug on a condition associated with expression of CXCR4 receptors, such as, but not limited to, cancer. In exemplary embodiments, such methods include administering an amount of the drug to a host with cancer, administering a detectably effective amount of a composition of ¹⁸F-TN14003 to a host, creating a radiographic image of the location and distribution of the ¹⁸F-TN14003 in the host with an imaging apparatus, and determining an amount of ¹⁸F-TN14003 taken up by host cancer cells. The amount of uptake of ¹⁸F-TN14003 by the host is related to the effect of the drug for treating cancer in the host.

The present disclosure also provides methods of synthesizing ¹⁸F-TN14003. In an exemplary embodiment, the synthesis includes providing N-hydroxysuccinimide ester of hydrazinonicotinic acid (NHS-HYNIC), mixing the NHS-HYNIC with TN14003 to form TN14003-HYNIC, mixing the TN14003-HYNIC with [¹⁸F]-fluorobenzaldehyde ([¹⁸F]FBA) to form ¹⁸F-TN14003-HYNIC (¹⁸F-TN14003), and separating ¹⁸F-TN14003 from unreacted TN14003-HYNIC and [¹⁸F]FBA to obtain substantially pure ¹⁸F-TN14003. In particular embodiments, the yield of ¹⁸F-TN14003 can be increased by varying the ratio of TN14003-HYNIC and [¹⁸F]FBA.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### BRIEF DESCRIPTION OF THE DRAWINGS

Aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

The patent or application file contains at least one drawing executed in color. Copies of this patent or patent application publication with color drawing(s) will be provided by the Office upon request and payment of the necessary fee.

FIG. 1 illustrates the specificity of the CXCR4 antagonist. FIG. 1A is a scanned image illustrating that the binding of the antagonist to CXCR4 was blocked by preincubation of 400 ng/ml of SDF-1. Cells were immunostained by using biotin labeled control peptide (left) or biotin-labeled CXCR4 antagonist (center and right) and streptavidin-conjugated rhodamine. Cells were preincubated with SDF-1 for 10 min and then fixed in ice-cold acetone (right). Original magnification was ×200. FIG. 1B is a scanned image of Northern blot analysis and Western blot analysis results of MDAMB-231 and MDAMB-435. FIG. 1C is a scanned image of confocal micrographs of CXCR4 protein on cell's surface from MDA-MB-231 and MDA-MB-435 cell lines by using biotinylated CXCR4 antagonist. Nuclei were counter-stained by cytox blude. Original magnification was ×100. FIG. 1D is a graph illustrating the quantitation of CXCR4 expression on MDA-MB-231 and MDA-MB-435 cell lines by flow cytometer. FIG. 1E is a scanned image of representative immunofluorescence staining of CXCR4 with the bitotinylated CXCR4 antagonist on paraffin-embedded tissue sections of breast cancer patients and normal breast tissue.

FIG. 2 illustrates that CXCR4 antagonists block tumor malignancy in head and neck cancer. FIG. 2A is a scanned image of results of Northern Blot analysis illustrating that CXCR4 mRNA levels in metastatic clones were significantly higher than those in non-metastatic parental clones. Subclones of squamous cell carcinoma of the head and neck (SCCHN) cell line 686LN were generated by passing these cells through several serial metastases in the SCCHN orthotopic animal model. The sub-populations of 686LN cells included one group with elevated metastatic activity and another group with non-metastatic parental cells. FIG. 2B is a scanned image of an FDG-PET taken from animals injected with metastatic 686LN cells intravenously, showing that the injected animals exhibit lung metastases. In this animal model, TN14003 treatment inhibited lung metastasis. FIG. 2C is a scanned image of bioluminescence imaging of orthotopic xenografts of head and neck cancer treated with TN 14003. Metastatic 686LN cells (5×10⁵) were injected into the neck area of nude mice, and they were divided into two groups, one with TN14003 treatment and the other with control peptide injection. 1 mg/kg of TN14003 was injected three times weekly i.p. TN14003 treatment started after tumors were established (day 8). Primary tumors were immunostained by using anti-CD31 antibody, and the microvessel density was calculated by averaging CD31-positive microvessels of primary tumors from each group (n=4) as illustrated in the bar graph of FIG. 2D. FIG. 2E is a scanned image of representative H & E stainings (original magnification, ×10) from two mice from each group.

FIGS. 3A and 3B illustrate that the interaction between CXCR4 and AMD3100 small molecule (FIG. 3A) is limited by two aspartic acids (171 and 262), while that between CXCR4 and TN14003 (ligand-mimicking peptide) (FIG. 3B) is through multiple interactions (Asp171, Phe174, ARG188, TYR190, Phe201, Gly207, Asp262).

FIG. 4A illustrates the structure of TN14003-Biotin. FIG. 4B shows scanned images illustrating a competitive binding assay using TN14003 as a tool for drug screening. 20,000 cells of MDA-MB-231 were seeded in 8-well slide chamber two days before experiments. Various concentration of the selected compounds (10,100, and 1000 nM) were added to the separate wells, incubated for 10 minutes at room temperature, and the cells were fixed in 4% of ice-cold paraformaldelyde. The slides were subsequently incubated with 50 ng/ml of biotin-TN14003 and streptavidin-Rhomdamine, followed by sytox blue. CXCR4—Red, nuclei-blue (sytox blue)

FIG. 5A is a bar graph illustrating inhibition of CXCR4/SDF-1 mediated invasion of MDA-MB-231 in vitro by WZ811 S (a novel compound developed through the drug screen using TN14003 as a tool) compared to TN14003 and AMD3100. Cells were seeded on top of the matrigel and added SDF-1 to the lower chamber. Invasive cells penetrate matrigel and end up on the other side of the matrigel. Invasion was estimated by counting the number of invading cells stained by H & E at the bottom side of the matrigel chamber and setting the average of invading cell numbers of MDA-MB-231 with SDF-1 added to the lower chamber as 100%. FIG. 5B is a scanned image that illustrates salt form of 6-18-10, WZ811S blocks SDF-1 induced the endothelial tubular formation in HUVECs. HUVECs were incubated with the presence of SDF-1 for 18 hours. HUVECs containing SDF-1 without the antagonist treatment formed excellent tubular networks. TN14003 or WZ811S pretreatment inhibited tubular network formation, whereas AMD3100 could not (P<0.001).

FIGS. 6A and 6B are graphs illustrating LANCE cAMP assay results of WZ811S counteracting SDF-1 induced increase of absorption at 665 nm (A665) that correlates to the reduction of cAMP. FIG. 6A illustrates that without WZ40MS absorption at 665 nm increased with an increasing concentration of SDF-1. SDF-1 was selected to be 30 ng/ml (˜EC₈₀) for 6B. FIG. 6 B illustrates that 100 percent in y-axis is the maximum A₆₆₅ induced by 30 ng/ml of SDF-1α (4.2 nM) (as indicated in 6A), and WZ811 S reduced SDF-1-induced absorption at 665 nm at a dose dependent manner. These figures demonstrate the ability to use the CXCR4 antagonist TN14003 as a tool to screen drug candidates that will interfere CXCR4 function.

FIG. 7 is a schematic representation of a method of making ¹⁸F-labeled CXCR4 antagonist adapted from Ackerman's method.

FIG. 8 is a schematic representation of a method of generating fluorine-labeled CXCR4 antagonist (TN14003) adapted from Poethko et al.

FIG. 9 is a schematic representation of an embodiment of a method of generating ¹⁸F-labeled CXCR4 antagonist (TN14003) adapted from Poethko et al. and Chang et al.

FIG. 10 illustrates ¹H-NMR results of NHS-HYNIC. The peak assignment is indicated on chemical structure of HYNIC by letters a-f.

FIG. 11A illustrates sub-fractions of a mixture of TN14003-HYNIC and FBA. After the conjugation steps, the mixture was fractionated with increasing acetonitrile concentration in a mobile phase. Mass spectroscopy data confirmed that fraction 6 contained TN14003-HYNIC, while the fraction 11 contained 19F-labeled TN14003 (final product). Fractions 6 and 9 are indicated by arrows on the graph. FIG. 11B shows scanned images of immunofluorescence of CXCR4 using biotin-labeled TN14003. This illustrates that that the final product, ¹⁹F-labeled TN14003 preincubation completely blocked the binding of biotin-labeled TN14003 to CXCR4 on cell surface, demonstrating that its blocking efficacy was similar to unlabeled, original TN14003.

FIG. 12 is a schematic representation of a further optimized method of generating ¹⁸F-labeled CXCR4 antagonist (TN14003) from FIG. 9.

FIG. 13 illustrates a thin layer chromatography (TLC) radiogram (50% ethylacetate/50% Hexane) showing the successful separation of ¹⁸F— TN14003 from [¹⁸F]FBA by reverse-phase HPLC. (ACN=acetonitrile).

FIG. 14 illustrates a TLC radiogram showing the improved conjugation, illustrating almost 90% yield when the ratios of TN14003-HYNIC to [¹⁸F]FBA were varied to optimize reactivity.

FIG. 15 illustrates a mass spectrograph illustrating that ¹⁸F-TN14003 was successfully separated from [¹⁸F]FBA and unlabeled TN14003-HYNIC.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere. Embodiments of the present disclosure will employ, unless otherwise indicated, techniques of medicine, pharmacology, nuclear chemistry, biochemistry, molecular biology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Prior to describing the various embodiments, the following definitions are provided and should be used unless otherwise indicated.

Definitions:

The term “CXCR4 antagonist” refers to a substance including, but not limited to, a polypeptide, polynucleotide, inhibitory polynucleotide, or siRNA, that interferes or inhibits the biological activity of the CXCR4 receptor including, but not limited to, the binding of a ligand to the receptor. Exemplary CXCR4 antagonists include, but are not limited to, TN14003, TC14012, and TE14011, and siRNAs directed to the CXCR4 receptor.

The term “CXCR4 peptide antagonist” refers to a polypeptide that specifically binds to CXCR4, particularly polypeptides that are not an antibody. Representative CXCR4 peptide antagonists include T140 and derivatives of T140. Exemplary derivatives of T140 include, but are not limited to, TN14003, TC14012, and TE14011 as well as those found in Tamamura, H. et al. Synthesis of potent CXCR4 inhibitors possessing low cytotoxicity and improved biostability based on T140 derivatives, Org. Biomol. Chem. 1:3656-3662, 2003, which is incorporated by reference herein in its entirety.

As used herein, the term “imaging probe”, “imaging agent”, or “imaging compound” refers to the radiolabeled compounds of the present disclosure that are capable of serving as imaging agents and whose uptake is related to the expression level of certain surface cell receptors, particularly CXCR4 receptors. In particular embodiments the imaging probes or imaging agents of the present disclosure are labeled with a PET isotope, such as F-18.

The term “peptide,” “polypeptides,” and “protein” include proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (IIe, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide that differs from a reference polypeptide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within 2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: GIn, His), (Asp: Glu, Cys, Ser), (GIn: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: lie, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

As used herein “functional variant” refers to a variant of a protein or polypeptide that can perform the same functions or activities as the original protein or polypeptide, although not necessarily at the same level (e.g., the variant may have enhanced, reduced or changed functionality, so long as it retains the basic function).

“Identity,” as known in the art, is a relationship between two or more polypeptide sequences, as determined by comparing the sequences. In the art, “identity” also refers to the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide. In the present application the terms “polypeptide” and “peptide” are used interchangeably, unless indicated otherwise.

A “pharmaceutically acceptable carrier” refers to a biocompatible solution, having due regard to sterility, pH, isotonicity, stability, and the like and can include any and all solvents, diluents (including sterile saline, Sodium Chloride Injection, Ringer's Injection, Dextrose Injection, Dextrose and Sodium Chloride Injection, Lactated Ringer's Injection and other aqueous buffer solutions), dispersion media, coatings, antibacterial and antifungal agents, isotonic agents, and the like. The pharmaceutically acceptable carrier may also contain stabilizers, preservatives, antioxidants, or other additives, which are well known to one of skill in the art, or other vehicle as known in the art.

As used herein, “pharmaceutically acceptable salts” refer to derivatives of the disclosed compounds wherein the parent compound is modified by making non-toxic acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, malefic, hydroxymaleic, phenylacetic, glutamic, benzoic, salicylic, mesylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like.

The pharmaceutically acceptable salts of the present disclosure can be synthesized from a parent compound that contains a basic or acidic moiety by conventional chemical methods. Generally, such salts can be prepared by reacting free acid forms of these compounds with a stoichiometric amount of the appropriate base (e.g., Na, Ca, Mg, or K, hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts may be found, e.g., in Remington's Pharmaceutical Sciences, 17th ed., Mack Publishing Company, Easton, Pa., p. 14¹⁸F (1985), which is hereby incorporated by reference in relevant part.

By “administration” is meant introducing a compound of the present disclosure into a subject. The preferred route of administration of the compounds is intravenous. However, any route of administration, such as oral, topical, subcutaneous, peritoneal, intraarterial, inhalation, vaginal, rectal, nasal, introduction into the cerebrospinal fluid, or instillation into body compartments can be used.

As used herein, the term “inhibit” and/or “reduce” generally refers to the act of reducing, either directly or indirectly, a function, activity, or behavior relative to the natural, expected, or average or relative to current conditions.

As used herein, the term “host”, “organism”, “individual” or “subject” includes humans, mammals (e.g., cats, dogs, horses, etc.), living cells, and other living organisms. A living organism can be as simple as, for example, a single eukaryotic cell or as complex as a mammal. “Patient” refers to an individual or subject who has undergone, is undergoing, or will undergo treatment.

In accordance with the present disclosure, “a detectably effective amount” of the imaging agent of the present disclosure is defined as an amount sufficient to yield an acceptable image using equipment that is available for clinical use. A detectably effective amount of the imaging agent of the present disclosure may be administered in more than one injection. The detectably effective amount of the imaging agent of the present disclosure can vary according to factors such as the degree of susceptibility of the individual, the age, sex, and weight of the individual, idiosyncratic responses of the individual, the dosimetry, and the like. Detectably effective amounts of the imaging agent of the present disclosure can also vary according to instrument and film-related factors. Optimization of such factors is well within the level of skill in the art.

The term “therapeutically effective amount” as used herein refers to that amount of the compound being administered which will relieve to some extent one or more of the symptoms of the disorder being treated. In reference to cancer or pathologies related to unregulated cell division, a therapeutically effective amount refers to that amount which has the effect of (1) reducing the size of a tumor, (2) inhibiting (that is, slowing to some extent, preferably stopping) aberrant cell division, for example cancer cell division, (3) preventing or reducing the metastasis of cancer cells, and/or, (4) relieving to some extent (or, preferably, eliminating) one or more symptoms associated with a pathology related to or caused in part by unregulated or aberrant cellular division, including for example, cancer, or angiogenesis.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to cancer, these terms also mean that the life expectancy of an individual affected with a cancer will be increased or that one or more of the symptoms of the disease will be reduced.

“Cancer”, “tumor”, and “precancerous” as used herein, shall be given their ordinary meaning, as general terms for diseases in which abnormal cells divide without control. Cancer cells can invade nearby tissues and can spread through the bloodstream and lymphatic system to other parts of the body. Various forms of cancer are discussed in greater detail below. It should be noted that cancerous cells, cancer, and tumors are sometimes used interchangeably in the disclosure.

Some abbreviations used throughout the disclosure include the following: CXCR4, CXC Chemokine receptor4; SDF-1; 18F or ¹⁸F, fluorine-18; stromal-derived factor-1; FACS, fluorescence-activated cell sorter; VEGF, vascular endothelial growth factor; MTT, methylthiazoletetrazolium; RT-PCR, Reverse transcription Polymerase Chain Reaction; MAb, monoclonal antibody; PE, R-Phycoerithrin; SCID, Severe Combined Immunodeficient; CC₅₀, 50% cytotoxic concentration; EC₅₀, 50% effective concentration; Si, selective index (CC₅₀/EC₅₀); DCIS, Ductal carcinoma in situ, H&E, hematoxylin and eosin; siRNA, small interfering RNA; HPRT, hypoxanthine-guanine-phosphoribosyltransferase.

Discussion:

Generally, the disclosure provides compositions and methods for imaging the expression, and particularly the overexpression, of certain surface cell receptors that are indicators of a disease or condition. In particular, the present disclosure relates to compositions and methods for imaging the expression of CXCR4 chemokine receptors for imaging conditions/diseases associated with CXCR4 receptor expression, including, but not limited, to inflammation, cancer, angiogenesis, tumors, and metastasis. Embodiments of the present disclosure include compositions and methods for the detection and staging of cancer and/or tumors and the prediction and/or diagnosis of metastasis. In embodiments, the present disclosure provides compositions and methods for imaging CXCR4 mediated pathology (e.g., cancer, angiogenesis, inflammation, and metastasis) by administering a labeled CXCR4 antagonist to a host in a detectably effective amount, for example in an amount sufficient to detect a cell expressing a CXCR4 receptor or homologue thereof. In particular, the CXCR4 antagonist is a peptide antagonist, and the peptide antagonist is not an antibody. Another embodiment provides uses of a CXCR4 antagonist and a radioisotope for the manufacture of an imaging agent for the imaging and staging of CXCR4 mediated pathologies including, but not limited to, cancer and tumor metastasis. Still another embodiment provides uses of a radiolabeled CXCR4 peptide antagonist for an imaging agent for the detection and prediction of tumor cell metastasis in a mammal.

The CXCR4 antagonists of the present disclosure include those described in detail in co-pending U.S. patent application Ser. No. 10/550,525, from which this application claims priority, and which is incorporated by reference above. Some of the peptide antagonists will be described below in greater detail. The CXCR4 antagonists of the present disclosure are labeled with a radiolabel suitable for imaging with gamma, PET or SPECT imaging technology, preferably an isotope suitable for PET imaging. Exemplary compositions described here can be used to image, detect, and/or predict cancer, in particular the spread of cancer, within an organism.

Cancer is a general term for diseases in which abnormal cells divide without control. Cancer cells can invade nearby tissues and can spread through the bloodstream and lymphatic system to other parts of the body. It has been discovered that the expression of CXCR4 receptors by cancer cells is a strong indicator of the metastatic potential of such cells. It has also been demonstrated that the administration of a CXCR4 antagonist to a host, for example a mammal, inhibits or reduces the metastasis of tumor cells, in particular breast cancer and prostate cancer.

There are several main types of cancer, and the disclosed compositions can be used to treat any type of cancer. For example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. A solid tumor is an abnormal mass of tissue that usually does not contain cysts or liquid areas. A single tumor may even have different populations of cells within it with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors. The compositions described herein can be used to image, detect, and follow the progression of tumor cells and their metastases, and thereby assist in the diagnosis and treatment of the cancer.

Representative cancers that may treated with the disclosed compositions and methods include, but are not limited to, bladder cancer, breast cancer, colorectal cancer, endometrial cancer, head & neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, ependymoma, Ewing's sarcoma family of tumors, germ cell tumor, extracranial cancer, Hodgkin's disease, leukemia, acute lymphoblastic leukemia, acute myeloid leukemia, liver cancer, medulloblastoma, neuroblastoma, brain tumors generally, non-Hodgkin's lymphoma, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, small-cell lung cancer, among others (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., inc., United States of America).

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a very different environment, not normally conducive to their growth) and continue their rapid growth and division in this new location. This is called metastasis. Once malignant cells have metastasized, achieving cure is more difficult. CXCR4 receptor antagonists are shown herein to be useful for the detection and prediction of metastasis of cancer cells.

Benign tumors have less of a tendency to invade and are less likely to metastasize. They do divide in an uncontrolled manner, though. Depending on their location, they can be just as life threatening as malignant lesions. An example of this would be a benign tumor in the brain, which can grow and occupy space within the skull, leading to increased pressure on the brain. Since CXCR4 is also produced to some extent by all tumors, but to a much greater extent by metastatic tumors, the compositions provided herein can be used to differentiate malignant and benign tumors.

CXCR4 Receptor and CXCR4 Receptor Antagonists

CXCR4 is a G-coupled heptahelical receptor which first drew attention as a major coreceptor for the entry of HIV. Activation of CXCR4 by SDF-1 results in activation of many downstream pathways including MAPK, PI3K, and calcium mobilization (Bleul, C. C. et al. The lymphocyte chemoattractant SDF-1 is a ligand for LESTR/fusin and blocks HIV-1 entry. Nature, 382:829-833, 1996; Deng, H. K. Expression cloning of new receptors used by simian and human immunodeficiency viruses. Nature, 355: 296-300, 1997; Vlahakis, S. R. et al. G protein-coupled chemokine receptors induce both survival and apoptotic signaling pathways. J. Immunol., 169:5546-5554, 2002; Sotsios, Y. et al. The CXC chemokine stromal cell-derived factor activates a Gi-coupled phosphoinositide 3-kinase in T lymphocytes. J. Immunol., 163: 5954-5963, 1999; Kijowski, J. et al. The SDF-1-CXCR4 axis stimulates VEGF secretion and activates integrins but does not affect proliferation and survival in lymphohematopoietic cells. Stem Cells, 19:453-466. 2001; Rozmyslowicz, T. et al. T-lymphocytic cell lines for studying cell infectability by human immunodeficiency virus. Eur. J. Haematol., 67:142-151, 2001; Majka, M. Biological significance of chemokine receptor expression by normal human megakaryoblasts. Folia. Histochem. Cytobiol., 39: 235-244, 2001; Majka, M. et al. Binding of stromal derived factor-1 alpha (SDF-1 alpha) to CXCR4 chemokine receptor in normal human megakaryoblasts but not in platelets induces phosphorylation of mitogen-activated protein kinase p42/44 (MAPK), ELK-1 transcription factor and serine/threonlne kinase AKT. Eur. J. Haematol., 64: 164-172, 2000). For hematopoietic stem cell activation, CXCR4 triggers migration to the marrow (Wright, D. E. et al. Hematopoietic stem cells are uniquely selective in their migratory response to chemokines. J. Exp. Med., 195: 1145-1154, 2002; Voermans, C. et al. Migratory behavior of leukemic cells from acute myeloid leukemia patients. Leukemia, 16: 650-657, 2002; Cashman, J. et al. Stromal-derived factor 1 inhibits the cycling of very primitive human hematopoietic cells in vitro and in NOD/SCID mice. Blood, 99: 792-799, 2002; Spencer, A. et al. Enumeration of bone marrow homing haemopoietic stem cells from G-CSF— mobilised normal donors and influence on engraftment following allogeneic transplantation. Bone Marrow Transplant, 28: 1019-1022, 2001; Vainchenker, W. Hematopoietic stem cells. Therapie, 56: 379-381, 2001; Liesveld, J. L. et al. Response of human CD34+ cells to CXC, CC, and CX3C chemokines: implications for cell migration and activation. J. Hematother. Stem Cell Res., 10: 643-655, 2001; Lapidot, T. Mechanism of human stem cell migration and repopulation of NOD/SCID and B2 mnull NOD/SCID mice. The role of SDF-1/CXCR4 interactions. Ann. N.Y. Acad. Sci., 938: 83-95, 2001; Kollet, O. et al. T. Rapid and efficient homing of human CD34(+)CD38(−/low)CXCR4(+) stem and progenitor cells to the bone marrow and spleen of NOD/SCID and NOD/SCID/B2m(null) mice. Blood, 97: 3283-3291, 2001) and directs peripheral blood cells into the lymph nodes and spleen (Blades, M. C. et al. Stromal cell-derived factor 1 (CXCL12) induces human cell migration into human lymph nodes transplanted into SCID mice. J. Immunol., 168: 4308-4317, 2002). Together these results indicate that SDF-1/CXCR4, may play a “lock and key” function for directing cells to a variety of target organs. As CXCR4 is a major coreceptor for T-tropic HIV infection, a variety of compounds that target CXCR4 to prevent infection have been developed.

Recently, an animal model of bone metastasis was generated by the intercardiac injection of MDA-MB-231 cells into female SCID mice, Kang, et al., Multigenic Program Mediating Breast Cancer Metastasis to Bone. Cancer Cell. 2003 June; 3(6):53749. A subsequent microarray analysis on a sub-population of MDA-MB-231 cells with elevated metastatic activity isolated from the mouse showed that one of the six genes responsible for the metastatic phenotype was CXCR4. Over-expression of CXCR4 alone in original MDA-MB-231 cells significantly increased the metastatic activity of the cells. In samples collected from various breast cancer patients, Muller et al, Involvement of Chemokine Receptirs in Breast Cancer Metastasis. Nature. 2001; 410(6824):50-6 found that the level of expression of CXCR4 is higher in primary tumors relative to normal mammary gland or mammary epithelial cells. By contrast, SDF-1 is highly expressed in the most common destinations of breast cancer metastasis including the lymph nodes, lung, liver, and bone marrow. Current evidence suggests that the expression of CXCR4 on breast cancer cell surfaces may direct such cells to the sites that are known to express high levels of SDF-1. It has been shown that CXCR4 antibody treatment inhibits metastasis to regional lymph nodes while all isotype controls metastasized to the lymph nodes and lungs Muller et al, Involvement of Chemokine Receptirs in Breast Cancer Metastasis. Nature. 2001; 410(6824):50-6. These data indicate that neutralization of the interaction between CXCR4 and its ligand, SDF-1, by a CXCR4 antibody can significantly impair metastasis of breast cancer cells to the lymph nodes and lungs. Taken together with the data provided in the examples below, CXCR4 and SDF-1 appear to play critical roles in breast cancer and head and neck cancer metastasis, thus, detection and quantification of CXCR4 expression levels can help in the detection of cancer and metastasis, the prediction of metastasis, as well as in monitoring the progression of cancer and/or the effectiveness of treatment regimens.

Anti-CXCR4 antibodies are capable of decreasing breast cancer metastasis at high concentrations (25 μg/ml). However, antibody therapy may be limited by: (1) the difficulty and expense of commercial-scale production; (2) delivery problems caused by slow diffusion due to a large mass (150 kDa); and (3) exclusion of the monoclonal antibody from compartments like the blood/brain barrier. Tumor masses of 1 cc usually contain 100,000,000 cancer cells. Large molecules such as antibodies with molecular weights of 150 kDa cannot easily diffuse between cells inside these densely populated tumor masses. Moreover, large antibodies are slowly eliminated from the body, and are thus not ideal candidates for radioactive imaging compounds. For instance, the use of an antibody (150 kDa) or antibody fragment (F(ab′)₂, 30 kDa) as an imaging probe for PET is not practical because PET nuclides such as carbon-11 and fluorine-18 have short half-lives, 20 and 109 minutes, respectively, while an antibody or antibody fragment will take a significantly longer time (at least 48 hours) to reach the target site (tumor) and clear out of the blood and tissue. Thus, there is a need for alternative antagonists to CXCR4 for imaging CXCR4 expression levels for detection and monitoring of cancer and metastasis.

Peptide Antagonists

In various embodiments, the compounds recited in the disclosure are representative of the compounds that may be used diagnostically and/or therapeutically in formulations or medicaments for the diagnosis, staging, and treatment-monitoring of chemokine mediated pathologies. Embodiments of the disclosure provide imaging compositions and methods of imaging a CXCR4 mediated pathology, or a pathology mediated by a CXCR4 chemokine receptor, in a host in need of such treatment, by administering to the host a detectably effective amount of a radiolabeled CXCR4 peptide antagonist, or a pharmaceutically acceptable salt thereof. Exemplary CXCR4 mediated pathologies or pathologies mediated by a CXCR4 receptor include, but are not limited to, cancer, tumors, angiogenesis, metastasis of a tumor/cancer, and inflammation.

In a preferred embodiment, the CXCR4 antagonist is a CXCR4 peptide antagonist such as T140 or a derivative of T140 such as TN14003. The sequence of T140 is H-Arg-Arg—NaI—Cys-Tyr-Arg-Lys—DLys—Pro-Tyr-Arg—Cit—Cys-Arg—OH (SEQ ID No.: 1) wherein Cit is L-citrulline, NaI is L-3-(2-naphthyl)alanine, and a disulfide bond links the two Cys residues. The sequence of TN14003 is H-Arg-Arg—NaI—Cys-Tyr—Cit-Lys—DLys—Pro-Tyr-Arg—Cit—Cys-Arg—NH₂ (SEQ ID No.: 2), wherein Cit is L-citrulline, NaI is L-3-(2-naphthyl)alanine, and a disulfide bond links the two Cys residues. It will be appreciated that more than one peptide antagonist can be used in sequence or combination.

Representative CXCR4 peptide antagonists include, but are not limited to, TN14003, TC14012, TE 14011, T140, T22, and derivatives, pharmaceutically acceptable salts, or prodrugs thereof as well as those found in Tamamura, H. et al. Synthesis of potent CXCR4 inhibitors possessing low cytotoxicity and improved biostability based on T140 derivatives, Org. Biomol. Chem. 1:3656-3662, 2003, incorporated by reference in its entirety. CXCR4 peptide antagonists are known in the art. For example, Tamamura et al. (Tamamura, E. L. et al. Pharmacophore identification of a specific CXCR4 inhibitor, T140, leads to development of effective anti-HIV agents with very high selectivity indexes. Bioorg. Med. Chem. Lett., 10: 2633-2637, 2000; Tamamura, H., et al. N. Conformational study of a highly specific CXCR4 inhibitor, T140, disclosing the close proximity of its intrinsic pharmacophores associated with strong anti-HIV activity. Bioorg. Med. Chem. Left, 11: 359-362. 2001) reported the identification of a specific CXCR4 inhibitor, T140, a 14-residue peptide that possessed a high level of anti-HIV activity and antagonism of T cell line-tropic HIV-1 entry among all antagonists of CXCR4 (Tamamura, E. L. et al. Pharmacophore identification of a specific CXCR4 inhibitor, T140, leads to development of effective anti-HIV agents with very high selectivity indexes. Bioorg. Med. Chem. Lett., 10: 2633-2637, 2000; Tamamura, H. et al. Conformational study of a highly specific CXCR4 inhibitor, T140, disclosing the close proximity of its intrinsic pharmacophores associated with strong anti-HIV activity. Bioorg. Med. Chem. Lett, 11: 359-362, 2001; Tamamura, H. et al. A low-molecular-weight inhibitor against the chemokine receptor CXCR4: a strong anti-HIV peptide T140. Biochem. Biophys. Res. Commun., 253: 877-882, 1998) by mimicking SDF-1. Further improvements in the compound were achieved by amidating the C-terminal of T-140, and by reducing the total positive charges of the molecule by substituting basic residues with nonbasic polar amino acids. This resulted in the generation of a compound (TN14003) with properties which are far less cytotoxic and more stable in serum compared to T140 (Tamamura, H. Development of specific CXCR4 inhibitors possessing high selectivity indexes as well as complete stability in serum based on an anti-HIV peptide T140. Bioorg. Med. Chem. Lett, 11: 1897-1902, 2001). The concentrations of T140 and TN14003 required for 50% protection of HIV-induced cytopathogenicity in MT4 cells (EC₅₀) are 3.3 nM and 0.6 nM respectively. The concentrations of T140 and TN14003 that induce a 50% reduction of the viability of MT4 cells (CC₅₀) are 59 μM and 410 μM respectively. These results reflect the improved therapeutic index for TN14003 over T140 (SI_{TN14003}=680,000; SI_{T140}=17,879; SI=CC₅₀/EC₅₀). The sequence of T22 is RRWCYRKCYKGYCYRKCR (SEQ ID NO: 3).

The imaging compounds of the present disclosure for imaging chemokine related conditions include a CXCR4 peptide antagonist, such as those described above, and a label (e.g., a radiolabel) suitable for use in imaging technologies such as a gamma camera, a PET apparatus, a SPECT apparatus, and the like. In a particular embodiment, the CXCR4 antagonist is TN14003, which binds to the SDF-1 binding site of CXCR4 protein. Some exemplary embodiments of non-radioactive elements and their radioactive counterparts that can be used as labels in the imaging probes of the present disclosure include, but are not limited to, F-19 (F-18), C-12 (C-11), I-127 (1-125, I-124, I-131, I-123), CI-36 (CI-32, CI-33, CI-34), Br-80 (Br-74, Br-75, Br-76, Br-77, Br-78), Re-185/187 (Re-186, Re-188), Y-89 (Y-90, Y-86), Lu-177, and Sm-153. Preferred imaging probes of the present disclosure are labeled with one or more radioisotopes, preferably including ¹¹C, ¹⁸F, ⁷⁶Br, ¹²³I, ¹²⁴I, or ¹³¹I and more preferably ¹⁸F, ⁷⁶Br, or ¹²³I, ¹²⁴I or ¹³¹I and are suitable for use in peripheral medical facilities and PET clinics. In particular embodiments, the PET isotope can include, but is not limited to, ⁶⁴Cu, ¹²⁴I, ^{76/77}Br, ⁸⁶Y, ⁸⁹Zr, and ⁶⁸Ga. In an exemplary embodiment, the PET isotope is ¹⁸F. The data provided herein demonstrates that an ¹⁸F labeled CXCR4 antagonist binds to the SDF-1 binding site of CXCR4 protein and can be detected with a PET scanner.

Small molecule CXCR4 antagonists (including those described in the examples below) and polynucleotide CXCR4 antagonists (e.g., siRNA) are also described in co-pending U.S. patent application Ser. No. 10/550,525, which is incorporated above by reference. The methods of the present disclosure can also be modified by those of skill in the art to attach labels (e.g., PET isotopes) to such small molecule and polynucleotide antagonists for use in imaging according to the methods of the present disclosure.

Some embodiments of the imaging compositions of the present disclosure further include a pharmaceutically acceptable carrier and/or excipient. The use of and type of carrier will depend on the host and the mode of administration. If the imaging compositions are to be used in vitro (e.g., for imaging cells, tissue, and other samples) a pharmaceutically acceptable carriers may not be included. When used in vivo for imaging a host, various dosage forms may be used depending on the mode of administration to be employed. Exemplary dosage forms are described in greater detail below.

Methods of Use

The ability to noninvasively and quantitatively image conditions related to over-expression of certain cell surface receptors, such as CXCR4 chemokine receptors using radiolabeled antagonists provides methods of early detection of disease and monitoring of disease progression as well as monitoring the effectiveness of drugs and other treatments. For instance, in the case of CXCR4 receptors that are implicated in cancer as well as indicators of metastatic potential, imaging the expression of these receptors can assist in early and sensitive cancer detection and patient selection for clinical trials based on in vivo expression quantification as well as allow early tumor diagnosis and patient stratification, metastasis prediction and detection, and better treatment monitoring, dose optimization, and the like.

The data provided in the examples below demonstrates that CXCR4/SDF-1 interaction is one of the major requirements for head and neck cancer metastasis. The elevated level of CXCR4 in primary tumors correlates with the metastatic potential of tumors. CXCR4 overexpression has been found in other tumors, such as breast cancer (as discussed in U.S. patent application Ser. No. 10/550,525, incorporated by reference above), pancreatic cancer (Koshiba, T. et al. Expression of stromal cell-derived factor 1 and CXCR4 ligand receptor system in pancreatic cancer: a possible role for tumor progression. Clin. Cancer Res., 6: 3530-3535, 2000), ovarian epithelial tumors (Scotton, C. J. et al. Epithelial cancer cell migration: a role for chemokine receptors? Cancer Res., 61: 4961-4965, 2001), prostate cancer (Taichman, R. S. Use of the stromal cell-derived factor-1/CXCR4 pathway in prostate cancer metastasis to bone. Cancer Res., 62: 1832-1837, 2002), kidney cancer (Schrader, A. J. et al. CXCR4/CXCL12 expression and signalling in kidney cancer. Br. J. Cancer, 86: 1250-1256, 2002), and non-small cell lung cancer (Takanami, I. Overexpression of CCR7 mRNA in nonsmall cell lung cancer: correlation with lymph node metastasis. Int. J. Cancer, 105: 186-189, 2003).

Accordingly, embodiments of the present disclosure include methods of imaging breast, brain, pancreatic, ovarian, prostate, kidney, head and neck, and non-small lung cancer, among others, as well as methods for detecting/predicting the metastatic potential of such cancers. In particular, metastasis of breast, head and neck, brain, pancreatic, ovarian, prostate, kidney, and non-small lung cancer can be detected and/or predicted by administering a radiolabled CXCR4 peptide antagonist, such as TN14003, to host in need of such treatment in an effective amount, imaging the host with appropriate imaging technology (e.g., a PET scanner), and detecting the expression of CXCR4 receptors.

As also shown below and/or in U.S. patent application Ser. No. 10/550,525, neutralizing CXCR4/SDF-1 activation with the CXCR4 antibody impaired breast cancer metastasis to the lymph node and lung in animal models for breast cancer metastasis (Muller, A. Involvement of chemokine receptors in breast cancer metastasis. Nature, 410: 50-56, 2001), and similar results have been observed in prostate cancer bone metastasis. Additionally, a synthetic 14-mer peptide blocked the CXCR4 receptor binding to its ligand SDF-1 and inhibited CXCR4/SDF-1 mediated invasion in vitro and metastasis in vivo with a higher specificity than anti-CXCR4 antibodies (R & D Systems). The anti-invasion and anti-metastasis activity of this peptide correlated well with their inhibitory activity on SDF-1α binding to CXCR4. This antagonist is proven safe by proliferation assay, animal histology, and hemopoietic progenitor cell colony formation. Thus, the CXCR4 antagonist TN14003 may prove to be an effective therapeutic agent of breast cancer metastasis as well as inhibitors of T-tropic HIV infection.

The extent of cancerous disease (stage) is a major prognostic factor, and non-invasive staging using imaging technologies has a key role in design of treatment strategies (e.g., surgery vs. radio-chemotherapy vs. adjuvant chemotherapy). The radiolabeled compounds of the present disclosure accumulate in malignant cells to a substantially greater extent than in normal cells and accumulate in highly metastatic cells to a greater extent than in cancer or tumor cells that are not as likely to metastasize. Thus, administration of an imaging compound of the present disclosure is suitable for the identification and imaging of malignant cells and tumors and is further suitable for measuring the stage of tumor development and metastatic potential.

Yet another embodiment provides a method for predicting tumor cell metastasis in a mammal by administering a detectably effective amount of a labeled CXCR4 antagonist, for example a peptide antagonist, pharmaceutically acceptable salt, or prodrug thereof and determining the level of expression of CXCR4 receptors by the tumor, where a higher level of CXCR4 expression is associated with a greater potential for metastasis. Embodiments of the present disclosure also include monitoring the treatment of cancer or metastasis by tracking the expression of CXCR4 as an indicator of the effectiveness of the treatment.

The amount of imaging agent used for diagnostic purposes and the duration of the imaging study will depend upon the radionuclide used to label the agent, the body mass of the patient, the nature and severity of the condition being treated, the nature of therapeutic treatments which the patient has undergone, and on the idiosyncratic responses of the patient. Ultimately, the attending physician will decide the amount of imaging agent to administer to each individual patient and the duration of the imaging study.

The present disclosure also includes methods of determining the effectiveness of a drug on various conditions associated with expression (particularly overexpression) of CXCR4 chemokine receptors. Conditions that can be monitored with respect to drug effectiveness include, but are not limited to, inflammation, cancer, tumors, angiogenesis, and metastasis. For instance, the methods of the present disclosure can be used to determine whether a particular drug is effective at inhibiting metastasis in a host having cancer, by monitoring the level of expression of CXCR4 receptors in the host cancer cells, which is an indicator of metastasis and metastatic potential. If expression of CXCR4 receptors decreases with drug treatment, that would indicate that the drug appears to be at least somewhat effective at inhibiting metastasis of the cancer/tumor in the host.

Such methods include administering an amount of a drug to a host; administering a detectably effective amount of a composition including a imaging probe including a radiolabeled CXCR4 antagonist (e.g., ¹⁸F-TN14003) or a pharmacutically acceptable salt thereof to a host; creating a radiographic image of the location and distribution of the imaging probe in the host with an imaging apparatus; and determining an amount of the imaging probe taken up by host cells wherein the amount of uptake by host mitochondria is related to the effect of the drug on apoptosis in host cells.

Embodiments of this disclosure include, but are not limited to: methods of imaging tissue; methods of imaging precancerous tissue, cancer, and tumors; methods of treating precancerous tissue, cancer, and tumors; methods of diagnosing precancerous tissue, cancer, and tumors; methods of monitoring the progress of precancerous tissue, cancer, and tumors; methods of imaging abnormal tissue, and the like. Embodiments of the present disclosure can be used to detect, study, monitor, evaluate, and/or screen, biological events in vivo or in vitro, such as, but not limited to, CXCR4 related biological events.

In general, as discussed above the radiolabeled compounds of the present disclosure can be used in vivo or in vitro for imaging cancer cells or tissue; imaging precancerous cells or tissue; diagnosing precancerous tissue, cancer, tumors, and tumor metastases; monitoring the progress and/or staging of precancerous tissue, cancer, and tumors; methods of predicting tumor metastasis; methods of evaluating drug effectivness on treating and/or preventing cancer, tumors, metastasis, and the like. Embodiments of the present disclosure can be used to detect, study, monitor, evaluate, and/or screen, biological events in vivo or in vitro, such as, but not limited to the expression of CXCR4 receptors. For example, the radiolabeled peptide antagonists of the present disclosure (as described above) can be provided to a host in an amount effective to result in uptake of the compound into the cells or tissue of interest. The host is then exposed to an appropriate PET or SPECT source (e.g., a light source) after a certain amount of time. The cells or tissue that take up the radiolabeled peptide antagonist can be detected using a PET or SPECT imaging system.

In diagnosing and/or monitoring the presence of cancerous cells, precancerous cells, and tumors in a subject, radiolabeled peptide antagonists are administered to the subject in an amount effective to result in uptake of the radiolabeled peptide antagonists into the cells. After administration of the radiolabeled peptide antagonists, cells that take up the radiolabeled peptide antagonists are detected using PET or SPECT imaging. Embodiments of the present disclosure can non-invasively image tissue throughout an animal or patient.

It should be noted that the amount effective to result in uptake of the compound into the cells or tissue of interest will depend upon a variety of factors, including for example, the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts.

Preferred imaging methods provided by the present disclosure include the use of the radiolabeled peptide antagonists of the present disclosure and/or salts thereof that are capable of generating at least a 2:1 target to background ratio of radiation intensity, or more preferably about a 5:1, about a 10:1 or about a 15:1 ratio of radiation intensity between target and background. In certain preferred methods, the radiation intensity of the target tissue is more intense than that of the background. In other embodiments, the present disclosure provides methods where the radiation intensity of the target tissue is less intense than that of the background. Generally, any difference in radiation intensity between the target tissue and the background that is sufficient to allow for identification and visualization of the target tissue is sufficient for use in the methods of the present disclosure.

In preferred methods of the present disclosure, the compounds of the present disclosure are excreted from tissues of the body quickly to prevent prolonged exposure to the radiation of the radiolabeled compound administered to the patient. Typically compounds of the present disclosure, including ¹⁸F-TN14003 and salts thereof, are eliminated from the body in less than about 24 hours. More preferably, compounds of the present disclosure are eliminated from the body in less than about 16 hours, 12 hours, 8 hours, 6 hours, 4 hours, 2 hours, 90 minutes, or 60 minutes. Typically, preferred compounds are eliminated in between about 60 minutes and 120 minutes.

Preferred compounds of the present disclosure are stable in vivo such that substantially all, e.g., more than about 50%, 60%, 70%, 80%, or more preferably 90% of the injected compound is not metabolized by the body prior to excretion.

Typical subjects to which compounds of the present disclosure may be administered will be mammals, particularly primates, especially humans. For veterinary applications, a wide variety of subjects will be suitable, e.g. livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g. mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine or tissue samples, or blood urine or tissue samples of the animals mentioned for veterinary applications.

Images can be generated by virtue of differences in the spatial distribution of the imaging agents that accumulate at a site having expression, and/or overexpression, of the CXCR4 receptors. The spatial distribution may be measured using any imaging apparatus suitable for the particular label, for example, a gamma camera, a PET apparatus, a SPECT apparatus, and the like. The extent of accumulation of the imaging agent may be quantified using known methods for quantifying radioactive emissions. A particularly useful imaging approach employs more than one imaging agent to perform simultaneous studies. Alternatively, the imaging method may be carried out a plurality of times with increasing administered dose of the pharmaceutically acceptable imaging composition of the present disclosure to perform successive studies using the split-dose image subtraction method, as are known to those of skill in the art.

Preferably, a detectably effective amount of the imaging agent of the present disclosure is administered to a subject. A detectably effective amount (as described above) of the imaging agent of the present disclosure may be administered in more than one injection. The detectably effective amount of the imaging agent of the present disclosure can vary according to factors such as the degree of susceptibility of the individual, the age, sex, and weight of the individual, idiosyncratic responses of the individual, the dosimetry, and the like. Detectably effective amounts of the imaging agent of the present disclosure can also vary according to instrument and film-related factors. Optimization of such factors is well within the level of skill in the art.

The amount of imaging agent used for diagnostic purposes and the duration of the imaging study will depend upon the radionuclide used to label the agent, the body mass of the patient, the nature and severity of the condition being treated, the nature of therapeutic treatments which the patient has undergone, and on the idiosyncratic responses of the patient. Ultimately, the attending physician will decide the amount of imaging agent to administer to each individual patient and the duration of the imaging study.

Pharmaceutical Compositions and Dosage Forms

Pharmaceutical compositions and dosage forms of the disclosure comprise a radiolabeled CXCR4 antagonist of the disclosure (e.g., a CXCR4 peptide antagonist (e.g., TN14003)) or a or pharmaceutically acceptable salt thereof pharmaceutically acceptable polymorph, solvate, hydrate, dehydrate, co-crystal, anhydrous, or amorphous form thereof. Specific salts of an antagonist of CXCR4 include, but are not limited to, sodium, lithium, potassium salts, and hydrates thereof.

Pharmaceutical compositions and unit dosage forms of the disclosure typically also comprise one or more pharmaceutically acceptable excipients or diluents. Advantages provided by specific compounds of the disclosure, such as, but not limited to, increased solubility and/or enhanced flow, purity, or stability (e.g., hygroscopicity) characteristics can make them better suited for pharmaceutical formulation and/or administration to patients than the prior art.

Pharmaceutical unit dosage forms of the compounds of this disclosure are suitable for oral, mucosal (e.g., nasal, sublingual, vaginal, buccal, or rectal), parenteral (e.g., intramuscular, subcutaneous, intravenous, intraarterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure will typically vary depending on their use. For example, a dosage form used in the acute treatment of a disease or disorder may contain larger amounts of the active ingredient, for example a radiolabeled CXCR4 antagonist or combinations thereof, than a dosage form used in the chronic treatment of the same disease or disorder. Similarly, a parenteral dosage form may contain smaller amounts of the active ingredient than an oral dosage form used to treat the same disease or disorder. These and other ways in which specific dosage forms encompassed by this disclosure will vary from one another will be readily apparent to those skilled in the art. See, e.g., Remington's Pharmaceutical Sciences, 18th ed., Mack Publishing, Easton, Pa. (1990).

Typical pharmaceutical compositions and dosage forms comprise one or more excipients. Suitable excipients are well known to those skilled in the art of pharmacy or pharmaceutics, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets or capsules may contain excipients not suited for use in parenteral dosage forms. The suitability of a particular excipient may also depend on the specific active ingredients in the dosage form. For example, the decomposition of some active ingredients can be accelerated by some excipients such as lactose, or when exposed to water. Active ingredients that comprise primary or secondary amines are particularly susceptible to such accelerated decomposition.

The disclosure further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate or organic acids. A specific solubility modulator is tartaric acid.

Like the amounts and types of excipients, the amounts and specific type of active ingredient in a dosage form may differ depending on factors such as, but not limited to, the route by which it is to be administered to patients. However, typical dosage forms of the compounds of the disclosure comprise a pharmaceutically acceptable salt of an antagonist of CXCR4, or a pharmaceutically acceptable polymorph, solvate, hydrate, dehydrate, co-crystal, anhydrous, or amorphous form thereof, in an amount of from about 10 mg to about 1000 mg, preferably in an amount of from about 25 mg to about 750 mg, and more preferably in an amount of from 50 mg to 500 mg.

Synthesis

The present disclosure also includes novel methods of synthesizing radiolabeled peptide antagonists. In particular, the disclosure provides methods of labeling peptide antagonists with ¹⁸F. While the examples below describe this method with respect to the CXCR4 peptide antagonist TN14003, one of skill in the art will recognize that this method can be used for a variety of small peptides, such as other CXCR4 peptide antagonists. Thus, while in the discussion that follows, the methods of synthesis will be described for ¹⁸F-TN14003, one of skill in the art would understand that with minor modifications the method could be used for other small peptides.

An exemplary embodiment of a method of synthesizing ¹⁸F-TN14003 includes first providing or synthesizing N-hydroxysuccinimide ester of hydrazinonicotinic acid (NHS-HYNIC) and mixing the NHS-HYNIC with TN14003 to form TN14003-HYNIC. The NHS-HYNIC can be prepared as described below. Briefly, N,N-dimethylalininobenzaldehyde is mixed with methylene chloride to produce a clear slightly greenish-yellow solution. Methyl trifluoromethanesulfonate is then added, resulting in an immediate color change to intense yellow. After stirring overnight, diethyl ether is added to obtain a crude product. Recrystallization from methylene chloride/diethyl ether produced a fine light yellow crystalline powder of NHS-HYNIC. In an embodiment, the NHS-HYNIC is mixed with the TN14003 by adding NHS-HYNIC solution in N,N-dimethylformamide (DMF) to TN14003 solution in 0.1 M sodium bicarbonate (pH 8.3). In embodiments, the molar ratio of HYNIC to TN14003 is about 1.5:1. After an incubation period, the TN14003-HYNIC is then separated from unreacted NHS-HYNIC with TN14003 by HPLC or other separation method. The TN14003-HYNIC can then be used immediately or stored for later use.

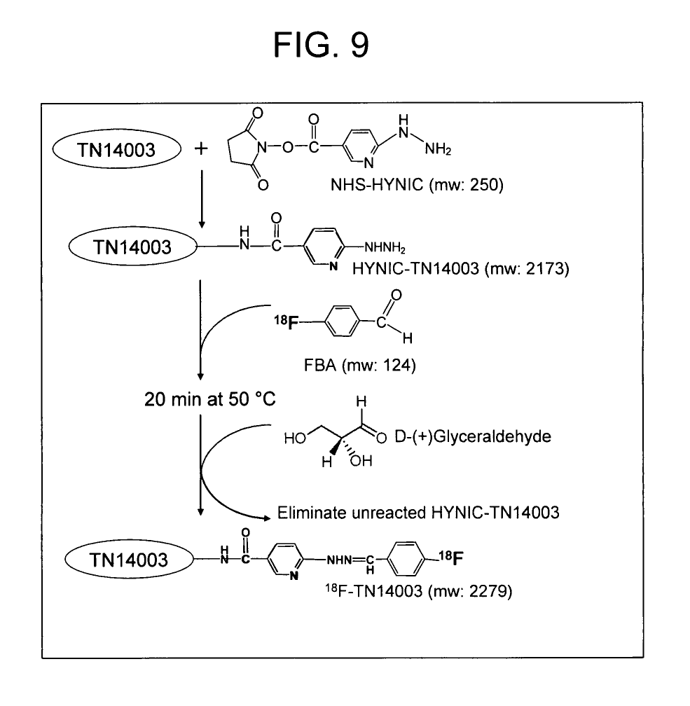

To radiolabel the TN14003-HYNIC with ¹⁸F, ¹⁸F-TN14003 TN14003-HYNIC is mixed with [¹⁸F]-fluorobenzaldehyde ([¹⁸F]FBA) to form ¹⁸F-TN14003-HYNIC (18F-TN14003). After reaction, the ¹⁸F-TN14003 is separated from unreacted TN14003-HYNIC and [¹⁸F]FBA to obtain substantially pure ¹⁸F-TN14003. This separation can be performed by reverse phase-HPLC. In an embodiment ¹⁸F-TN14003 is purified by reverse phase-HPLC to eliminate unconjugated TN14003-HYNIC. The hydrophilic aldehyde conjugated the HYNIC, [¹⁸F] is removed at the early phase (0.1% TFA in water), and TN14003-HYNIC is eliminated at the medium phase (12% acetonitrile, 0.1% TFA in water). The ¹⁸F-TN14003 is collected at about 20% acetonitrile and 0.1% TFA in water. The final product can be confirmed by mass spectroscopy. This synthesis method is illustrated in FIG. 9 and described in greater detail in the examples below.

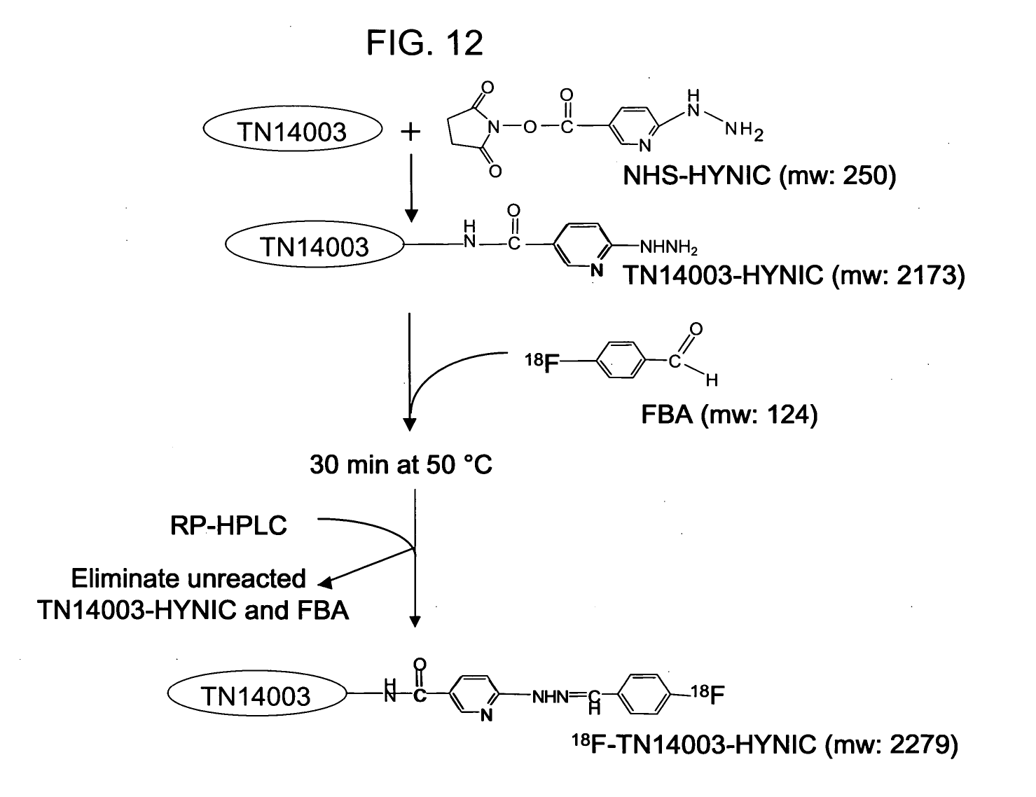

FIG. 12 illustrates a modified version of the above-described synthesis, in which the ¹⁸F-TN14003 is separated from unreacted TN14003-HYNIC and [¹⁸F]FBA by reverse phase HPLC using a C18 Sep-Pak with a gradient of acetonitrile. The [¹⁸F] was removed at the early phase (0.1% TFA in water) and unreacted TN14003-HYNIC was eliminated at the second phase (12% acetonitrile, 0.1% TFA in water). The fraction with the ¹⁸F-TN14003 was collected at the third phase (20% acetonitrile, 0.1% TFA in water). Then [¹⁸F]FBA gets eluted at the last phase with 40% acetonitrile in 0.1% TFA in water. This process is described in greater detail in the examples below.

Kits

The present disclosure also provides packaged pharmaceutical compositions comprising a pharmaceutically acceptable carrier and an imaging compound of the disclosure (e.g., ¹⁸F-TN14003). In certain embodiments, the packaged pharmaceutical composition includes the reaction precursors to be used to generate the imaging compound according to the present disclosure upon combination with a radiolabeled precursor. Other packaged pharmaceutical compositions provided by the present disclosure further include indicia including at least one of: instructions for using the composition to image a host, or host samples (e.g., cells or tissues) for expression of CXCR4 receptors, which can be used as an indicator of conditions including, but not limited to, cancer, a tumor, cancer progression, angiogenesis, inflammation, and metastasis. In embodiments, the kit may include instructions for using the composition to assess therapeutic effect of a drug protocol administered to a patient, instructions for using the composition to selectively image malignant cells and tumors, and instructions for using the composition to predict metastatic potential.

This disclosure encompasses kits that include, but are not limited to, radiolabeled CXCR4 peptide antagonists (e.g., TN14003) and directions (written instructions for their use). The components listed above can be tailored to the particular biological event to be monitored as described herein. The kit can further include appropriate buffers and reagents known in the art for administering various combinations of the components listed above to the host cell or host organism.

In certain preferred embodiments, the present disclosure provides a kit including from about 1 to 30 mCi of the radionuclide-labeled imaging agent described above (preferably ¹⁸F-TN14003) in combination with a pharmaceutically acceptable carrier. The imaging agent and carrier may be provided in solution or in lyophilized form. When the imaging agent and carrier of the kit are in lyophilized form, the kit may optionally contain a sterile and physiologically acceptable reconstitution medium such as water, saline, buffered saline, and the like.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

### EXAMPLES

Specificity of the CXCR4 Antagonist

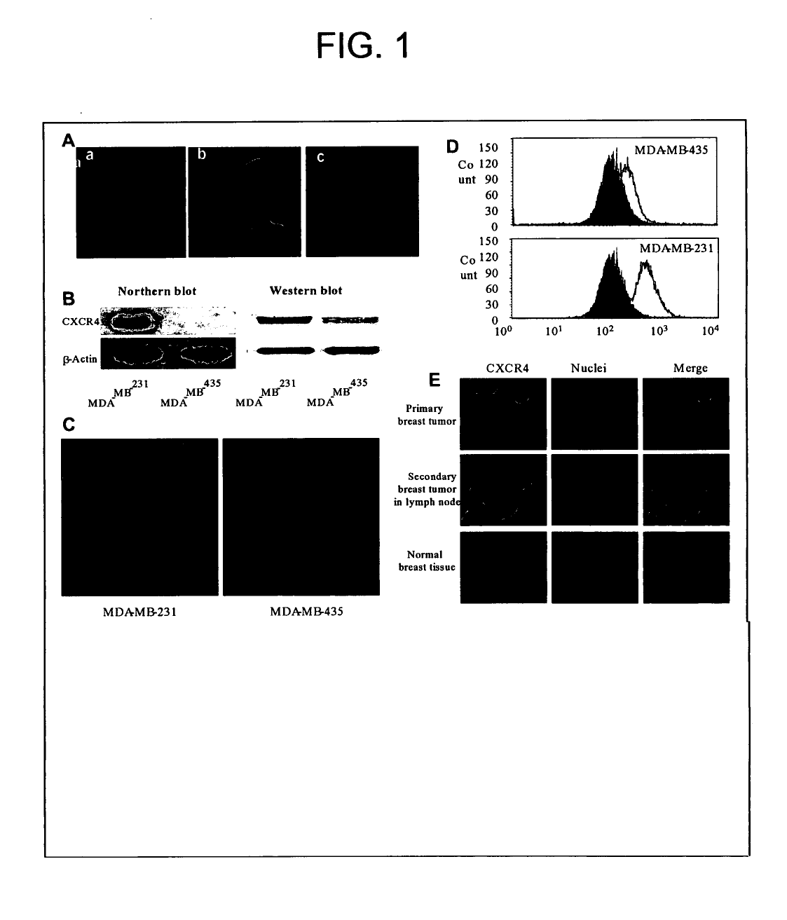

If CXCR4 activity can be minimized (or inhibited) in tumor cells, it might be possible to limit their ability to spread to other organs. This kind of preventive medicine will be especially beneficial to a group of patients who have a localized or pre-malignant tumor with a high expression of CXCR4. The ideal CXCR4 antagonist should bind to the SDF-1 binding site of the CXCR4 protein and block SDF-1 binding. Such an antagonist, TN14003, with high specificity and low toxicity was described in co-pending U.S. patent application Ser. No. 10/550,525, which is incorporated by reference above. The antagonist is a 14-mer, 2 kDa peptide, which is much smaller than the CXCR4 antibody. This synthetic peptide is stable for at least 36 hours in serum at 37° C. TN14003 can easily be labeled by biotin and used for immunofluorescence with streptavidin-conjugated fluorescence dyes such as fluorescein isothiocyanate (FITC) or phycoerythrin (PE), a property that is useful for detecting target proteins. To demonstrate that TN14003 binds to the SDF-1 binding site of CXCR4, MDA-MB-231 cells were pre-incubated with 400 ng/ml of SDF-1 and then added the biotin-labeled TN14003. As shown in FIG. 1A, SDF-1 binds to CXCR4 receptors on cells and blocks binding of the biotin-labeled TN14003. This demonstrates that TN14003 binds to the SDF-1 binding site of CXCR4 protein.

Utility of the CXCR4 Antagonist as an Imaging Probe

Initially, biotin-labeled CXCR4 antagonist, TN14003, was used as a probe for CXCR4 for both immunofluorescence staining and FACS analysis of cultured breast cancer cells and paraffin-embedded tissues from breast cancer patients. MDA-MB-231 cells had high levels of CXCR4 mRNA as shown by Northern blot (FIG. 1B). In contrast, MDA-MB-435 had low levels of CXCR4 mRNA. When the biotinylated TN14003 was used to visualize the CXCR4 receptors on the cell surface, the MDA-MD-231 were brightly stained (FIG. 1C), consistent with their high levels of CXCR4 mRNA. Conversely, binding of biotinylated TN14003 to MDA-MB-435 was dramatically lower, consistent with the reduced mRNA levels in these cells. Flow cytometry of the cell lines confirmed the histochemical findings. MDA-MD-435 showed limited binding of the biotinylated TN14003 (FIG. 1D) in contrast to the results with MDA-MD-231, which showed bright red staining. This difference was confirmed by Western blot analysis using a CXCR4 antibody (Ab-2, Oncogene, FIG. 1B). Ab-2 antibody was not suitable for immunohistochemistry, and the difference in CXCR4 levels could not be demonstrated by immunohistochemistry using commercially available antibodies from R&D systems (MAB170, 171, 172, 173). Therefore, the biotin-labeled TN14003 is more beneficial than CXCR4 antibodies in quantitatively detecting CXCR4 proteins on the cell surface.

Next, it was determined that biotin-labeled TN14003 could be used to detect CXCR4 protein in formalin-fixed, paraffin-embedded tissues. FIG. 1E shows that CXCR4 expression levels are low in normal tissues (no red rhodamine staining) while primary tumors and lymph node metastases from the same patient showed elevated CXCR4 protein levels. Based on these results, it was concluded that the biotin-labeled CXCR4 antagonist of the present disclosure can serve as novel-imaging probes that are highly specific for CXCR4.

Blocking CXCR4 Blocks Cancer Metastasis in Animal Models for SCCHN

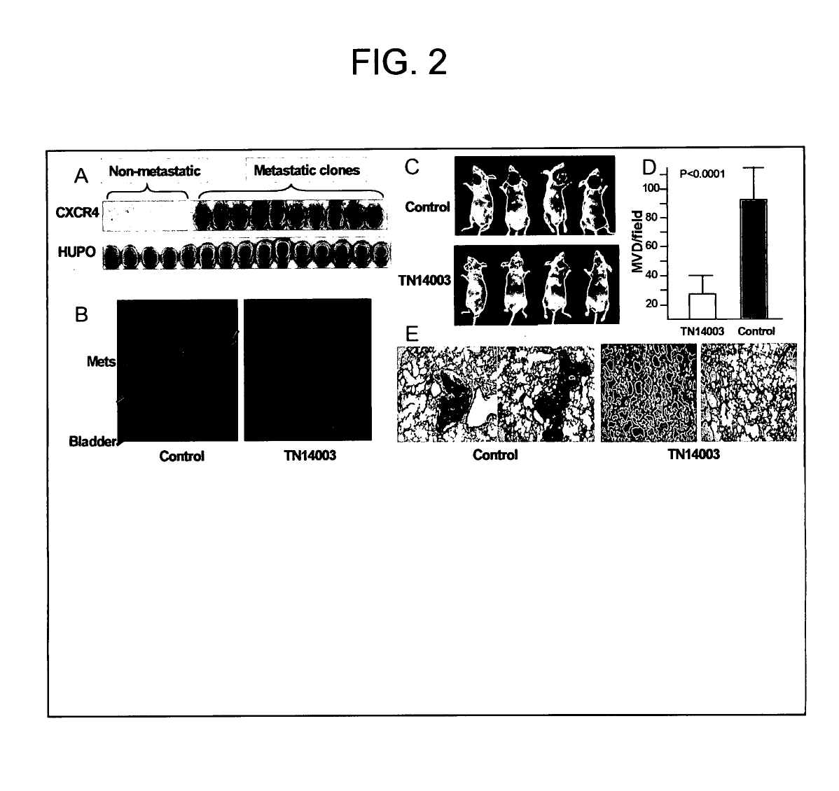

Metastatic SCCHN cell lines were established from a poorly metastatic 686LN parental cell line by four rounds of in vivo selection using a lymph node metastatic xenograft mouse model. It was observed that metastatic clones of SCCHN established from the same model expressed high levels of CXCR4 while non-metastatic parental clones established from the primary tumor of the same model did not (FIG. 2A). This result suggests that CXCR4 is required for the metastatic process. Therefore, the impact of blocking CXCR4 function on SCCHN progression in both orthotopic and experimental animal models was investigated to examine the role of CXCR4 in primary tumor growth and lung metastasis. The synthetic 14-mer peptide, TN14003 was utilized, which was shown in the co-pending U.S. patent application Ser. No. 10/550,525 (incorporated by reference above) to block the CXCR4 receptor by binding competitively with its ligand, SDF-1, and inhibit CXCR4/SDF-1 mediated invasion in vitro and metastasis in vivo with a higher specificity than the commercially available anti-CXCR4 antibodies (R & D Systems), (see also, Liang, et al, Inhibition of Breast Cancer metastasis by Selective Synthetic Polypeptide against CXCR4. Cancer Res. 2004 Jun. 15; 64(12):4302-8.). The anti-invasion and anti-metastasis activity of this peptide correlates well with its inhibitory activity on the binding of SDF-1 to CXCR4 in the MDA-MB-231 breast cancer cell line. It was also believed that TN14003 would block head and neck cancer metastasis. To demonstrate this, the metastatic cells were injected through the tail vein of the nude mice to create an experimental animal model for head and neck cancer metastasis and to determine the anti-metastatic efficacy of TN14003 in vivo by non-invasive FDG-PET imaging. FIG. 2B is a maximum intensity projection generated from six mice in each group. The chest area is significantly brighter in each mouse of the control group (left) than any of the mice in the TN14003-treated group (right), which indicates significantly more lung metastases in the control group as compared with those in the TN14003-treated group. The high FDG-uptake can also be seen in the bladder due to the secretion of FDG through the bladder. Lungs were collected from these animals and histological methods were used to confirm the results. Thus, blocking CXCR4 prevented lung metastasis of SCCHN.

The anti-tumor efficacy of the CXCR4 antagonist as also tested in an orthotopic SCCHN animal model. The metastatic cells were stably transfected with the luciferase gene (pGL₂-control from Promega) for in vivo tracking purposes. These cells (500,000 cells) were injected into the submandibular subcutaneous tissue to the mylohyoid muscle of the nude mice to create an orthotopic SCCHN xenograft, and the tumor growth was followed using non-invasive Bioluminescence Imaging (BLI). FIG. 2C shows the impact of the treatment of TN14003 on pre-established SCCHN of metastatic 686LN cells. The intraperitoneal treatment of TN14003 started 7 days after the tumor injection and lasted for 23 days compared to that of the control peptide. The BLI shows that TN14003 treatment (1 mg/kg, i.p.) suppressed even the primary tumor (FIG. 2C). To determine whether the suppression of primary tumor growth was due to an anti-angiogenic effect of the CXCR4 antagonist (e.g., the inhibition of the formation of microvessels of tumors), immunohistostaining of the tumor sections was performed with an anti-CD31 antibody. CXCR4/SDF-1 is known to play a critical role in tumor angiogenesis, which is crucial for tumor growth. As expected, a significant reduction of microvessel density (MVD) was observed in the tumors of CXCR4 antagonist-treated mice compared to those of the control group. CD31 immunostaining of primary tumors demonstrated that TN14003 significantly blocked tumor angiogenesis at the primary site and, thus, impacted the growth of the tumors (FIG. 2D). Tumor angiogenesis is a prerequisite for the spreading of cancer because the newly formed vasculatures provide a route for metastatic cells to travel to distant organ sites. SDF-1 has been reported to influence the secretion of vascular endothelial growth factor (VEGF) and vice versa, suggesting a mechanism for the role of CXCR4/SDF-1 in tumor angiogenesis.

Lungs were collected from these animals, sectioned into 6 μm slices, and every 10th slice was stained with H&E to locate the metastases (FIG. 2E). The two left panels show lung metastases from the mice of the control group, whereas the two right panels show the morphology of lungs from mice treated with TN14003. The images show a reduction in the metastases in the treated group as compared to the control group. Thus, blocking CXCR4 suppressed the primary tumors and prevented lung metastasis of head and neck cancer cells. In summary, these data demonstrate that CXCR4 is an excellent target for intervention in the process of tumor progression/metastasis.

Establishment and Optimization of Compound Screening Assays for Potent CXCR4 Antagonists

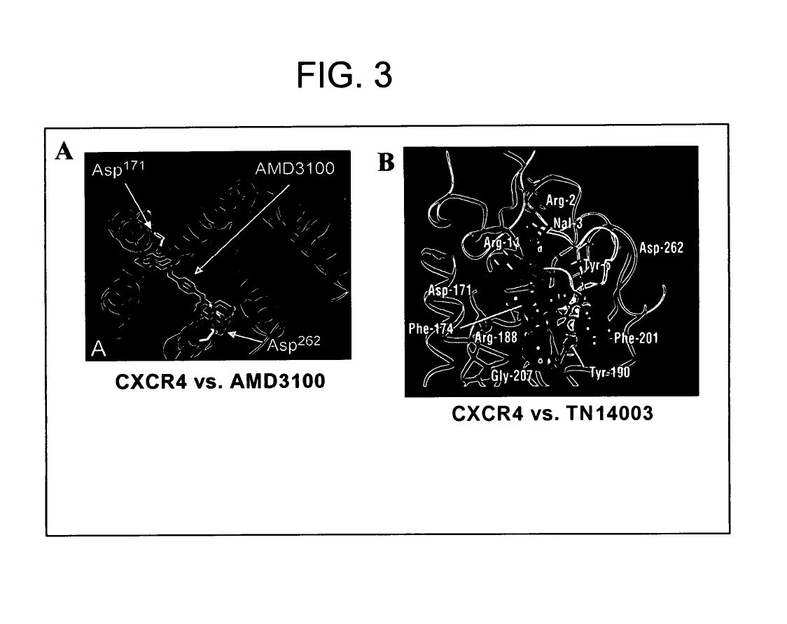

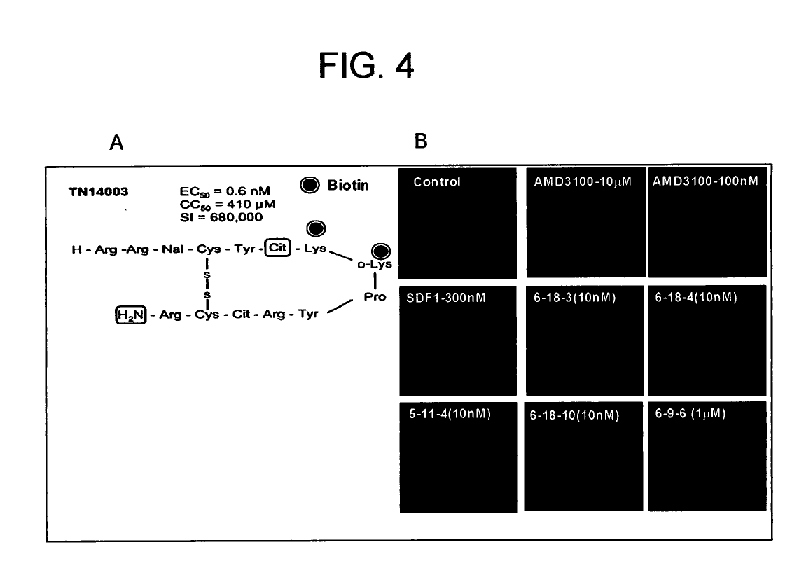

Screening of Small Molecule CXCR4 Antagonists With a Competitive Binding Assay Against Biotin-Labeled TN14003: T140 analogs, including TN14003, bind to the ligand binding site on CXCR4, blocking the CXCR4/SDF-1 interaction, and intervening in the progression of cancer metastasis. The discovery and development of effective, orally available small molecules also remains a major focus for many medicinal chemistry programs. Therefore, identification of a novel series of potent, small molecule antagonists could prove to be practical for preclinical advancement and progression into clinical evaluation. Currently, the metal-chelating cyclams and bicyclams represent the sole class of non-peptide molecules that are known to block CXCR4. One of these non-peptide molecules, AMD3100, was in clinical trials as an inhibitor of HIV cellular entry but was later withdrawn due to cardiotoxicity. Trent et al. employed the use of molecular modeling to understand the interactions with CXCR4 that are responsible for the antagonist activity of AMD3100. Trent, et al. Lipid Bilayer Simulations of XCXR4 with Inverse Agonists and Weal Partial Agonists. J. Biol. Chem. 2003 Nov. 21; 278(47):47136-44. Molecular dynamic simulations of the rhodopsin-based homology model of CXCR4 shows that AMD3100 interacts with CXCR4/SDF-1 binding through Asp171 and Asp262 (FIG. 3), and AMD3100 binding changes the orientation of the lower portions of the TM helices and cytoplasmic domains. The altered orientation provides a potential conformational rationalization for the finding that AMD3100 is a weak partial agonist. By contrast, the peptide-based CXCR4 antagonist, T140 (similar to TN14003), strongly binds the SDF-1 binding site of CXCR4 in extracellular domains and regions of the hydrophobic core proximal to the cell surface (multiple interactions with residues in CXCR4 (FIG. 3), including amino acids in the N-ter, TM4, E-L2, TM5, and E-L3). This information was used to create a library of compounds with multiple, basic nitrogens throughout the molecular framework that are structurally different from AMD3100. By using biotin-labeled TN14003 along with streptavidin-conjugated rhodamine, the binding efficiency of these chemicals to the SDF-1 binding site of CXCR4 on tumor cells was determined and compared to that of AMD3100 (FIG. 4). In this assay, cells incubated with compounds with high affinities for the ligand binding site showed only blue nuclei staining, whereas compounds with low affinity resulted in both CXCR4 in red (rhodamine) and blue nuclei staining (sytox blue). Cells were pre-incubated with different concentrations of AMD3100, and it was found that a 10 μM concentration was needed for AMD3100 to compete against biotin-labeled TN14003. In fact, Hatse et al. reported the IC₅₀ of AMD3100 to be 1-10 μM determined by calcium mobilization assay. Some of the novel compounds disclosed herein were as potent as TN14003 at very low concentrations (IC₅₀<10 nM). Hatse, et al. Chemokine Receptior Inhibition by AMD3100 is Strictly Confined to CXCR4. FEBS Lett. 2002 Sep. 11; 537(1-3):255-62. Initially, one of these compounds, 6-18-10, was selected to study its therapeutic potential based on its potency (IC₅₀<10 nM, FIG. 4 lower middle panel) and low toxicity to CXCR4-negative 2091 cells (cytotoxic index, CC₅₀>100 μM).

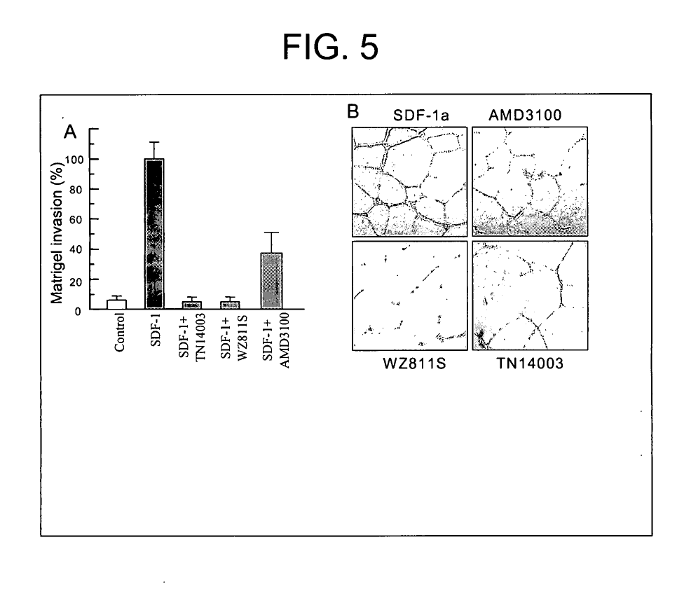

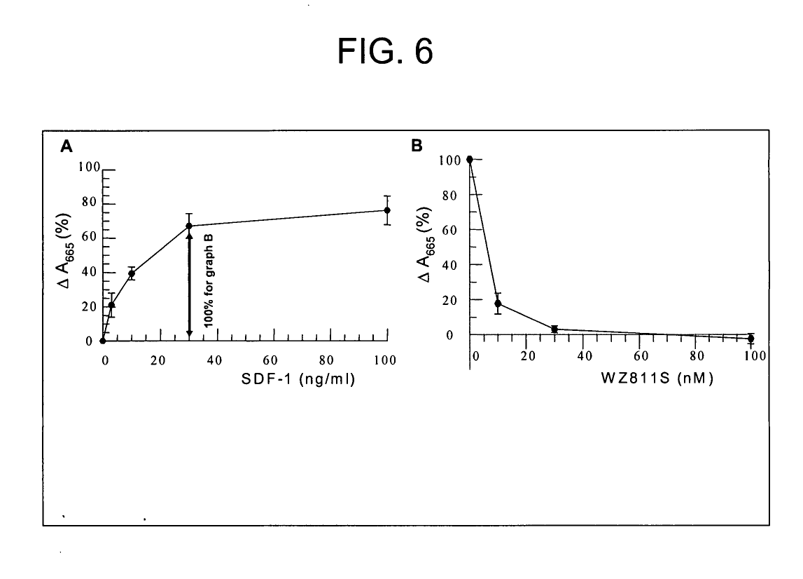

Functional Assays to Determine Anti-CXCR4 Efficacy of the Selected Compounds: TN14003 blocks SDF-1-mediated invasion more effectively than an anti-CXCR4 antibody in a matrigel invasion assay using SDF-1 as a chemoattractant (Liang et al.) Thus, this assay has been included into screening cascades to test the novel small molecules of the present disclosure, using TN14003 as a benchmark standard to control the assay. A salt form of 6-18-10, WZ811S, was tested in a matrigel invasion assay to measure its ability to inhibit CXCR4/SDF-mediated invasion. As shown in FIG. 5A, WZ811S was shown to be as potent as TN14003 in blocking SDF-1-induced invasion when tested at the same concentration (10 nM). AMD3100 was not as effective as WZ811S even at a ten-fold concentration (100 nM). Because the major pathway of CXCR4/SDF-1 is the pertussis toxin-sensitive Gi, it was more appropriate to use cAMP reduction as a direct measure of CXCR4/SDF-1-mediated Gαi than the calcium mobilization. Thus, Perkin-Elmer's LANCE cAMP assay kit (Cat # AD0262) that was based on time-resolved fluorescence resonance energy transfer (TR-FRET) was tested. The samples were prepared according to the manufacturer's instruction using 30 μM Forskolin to induce Gs-mediated cAMP production that was reduced by SDF-1. First, the absorption increase at 665 nm was determined by various concentrations of SDF-1 (0-100 ng/ml) to determine EC₈₀ to be 30 ng/ml (FIG. 6). With pre-treatment of WZ811S, the effect of 30 ng/ml of SDF-1 on cAMP reduction was significantly reduced at a dose dependent manner (FIG. 6). The results were measured in Perkin-Elmer's Envision multi-label microplate reader (384-wells) with conditions of flash energy area=low, flash energy level=239, counting cycle=1 ms, and ex/em=340 nm/665 nm. However, WZ811S had a short plasma half-life in mice (<10 mins) and could not block CXCR4-mediated metastasis as well as TN14003 even with more frequent injection schedule.

Developing TN14003 as an Imaging Probe for Non-Invasive PET Imaging

If CXCR4 is a required factor for metastasis, all malignant tumors should express high levels of CXCR4. Thus, it is believed that metastasis can be detected and/or predicted by CXCR4 expression levels. Unlike immunohistochemistry that detects over-expression of a target protein per cell; in vivo imaging detects the combination of (1) over-expression of a target protein per cell and (2) cell density effect. Therefore, it is possible to selectively detect CXCR4-positive solid tumors that have both high levels of CXCR4 and a greater cell density than normal tissues. Therefore, a CXCR4 antagonist was developed as an imaging probe for ¹⁸F-PET detection of CXCR4 over-expressing tumors with high metastatic potential. Between two CXCR4 antagonists, TN14003 and WZ811S (anti-CXCR4 small molecule under development), TN14003, a peptide-based CXCR4 antagonist, was selected as a PET imaging probe because the peptide-based CXCR4 antagonist has shown to strongly bind the SDF-1 binding site of CXCR4 in extracellular domains and regions of the hydrophobic core proximal to the cell surface (multiple interactions with residues in CXCR4 (FIG. 3), including amino acids in the N-ter, TM4, E-L2, TM5, and E-L3. Although small molecules are sometimes preferred as therapeutic drugs, their specificity/selectivity are not as good as ligand mimicking peptides with tightly binding multiple interaction sites. In addition, TN14003 was more effective than WZ811S in blocking metastasis in vivo. As previously demonstrated, three times weekly injection of TN14003 completely inhibited CXCR4-mediated cancer metastasis in vivo, while WZ811S could not.

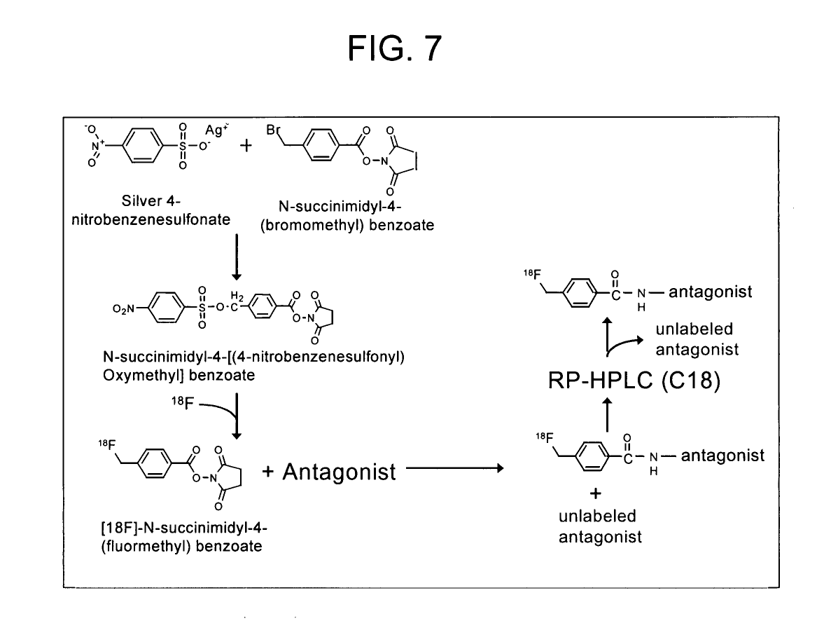

First, the fluorine labeling technique developed by Garg et al. Localization of Fluorine-18-labeled MeI-14 Monoclonal Antibody F(ab′)2 Fragment in a subcutaneous Xenograft Model. Cancer Res. 1992; 52(18):5054-60 and Lang et al. was adapted by mixing the ¹⁸F precursor, N-succinimidyl 4-[¹⁸F] (fluoromethyl) benzoate (SFB) with the CXCR4 antagonist (illustrated in FIG. 7). The linker made of ¹⁹F is exactly the same as the linker made of ¹⁸F except it carries no radioactivity. Therefore, an F19 linker was used to optimize the cross-linking conditions and elucidate the process using a MALDI-TOF Mass Spectrometer from the Emory School of Medicine Microchemical and Proteomics Core Facility. ¹⁹F—SFB (cold form) was used, and the conditions to label TN14003 and isolate the fluorine-labeled TN14003 from unlabeled TN14003 by reverse phase-HPLC (C18 Sep-Pak) were determined. Because the unlabeled TN14003 was more hydrophilic than the fluorine-labeled TN14003, the fluorine labeled peptide could be separated from the unlabeled peptide by washing the C18-Sep-Pak with a different concentration of acetonitrile. The carrier-free ¹⁹F-labeled TN14003 (no contamination with unlabeled TN14003) was confirmed by MALDI-TOF Mass Spectrometer at the Microchemical and Proteomics Core Facility (data not shown). Using biotin-labeled TN14003 along with streptavidin-conjugated FITC, FACS analysis was used to compare the binding efficiency of the fluorine-labeled TN14003 with that of the unlabeled TN14003 to CXCR4 receptor on tumor cells. Cells incubated with only streptavidin-conjugated FITC (negative control) were compared to cells incubated with both the biotin-labeled TN14003 and streptavidin-conjugated FITC (positive control). When equal amounts of unlabeled TN14003 were added into the biotin-labeled antagonist mixture, it was found that the FITC fluorescence decreased due to competition between biotin-labeled TN14003 and unlabeled TN14003. The same amount of FITC fluorescence decrease was seen when fluorine-labeled TN14003 was added to the biotin-labeled TN14003 mixture (data not shown). This demonstrates that the binding efficiency of fluorine-labeled antagonist is the same as that of unlabeled (original) antagonist. Therefore, fluorine labeling did not affect binding efficiency of TN14003 to the CXCR4 protein on the cell surface.

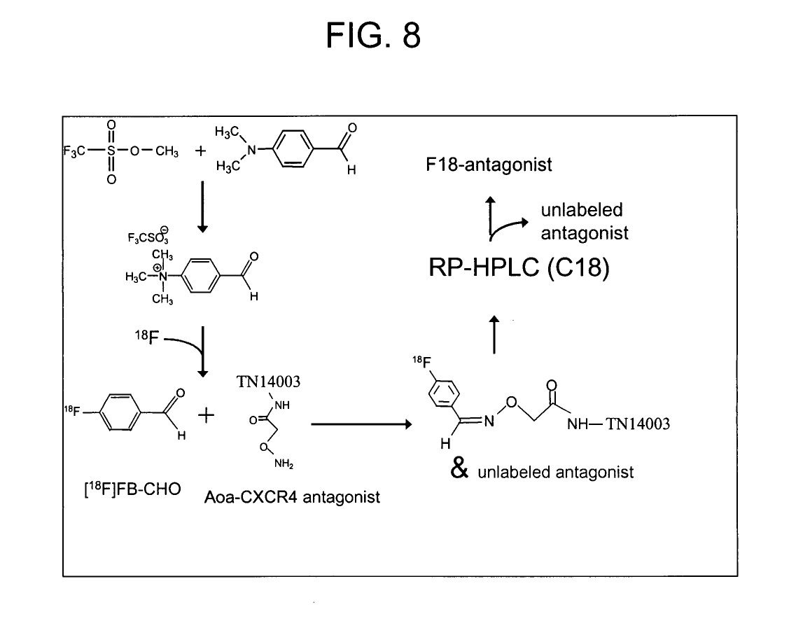

¹⁸F was generated from the on-site cyclotron within the Emory School of Medicine PET Center Core Facility, 20 mCi of ¹⁸F—SFB (10-12 moles) was made out of 1000 mCi of ¹⁸F (precursor yield was only 2%), and it was cross-linked with TN14003. The ¹⁸F-labeled TN14003 (15% yield) was isolated from unlabeled TN14003 using reverse phase HPLC (by using C18-Sep-Pak). The animals were injected with 150 μCi ¹⁸F-labeled TN14003 and, 60 minutes later, were sacrificed. The primary tumor and other organs were collected, then weighed and counted them for ¹⁸F activity using a gamma scintillation counter to determine the distribution of our compound in the mice. However, it was found that ¹⁸F dissociated from the ¹⁸F-TN14003 extremely quickly in vivo, indicated by extremely high radioactivity in bones (free ¹⁸F has high affinity for bone). Therefore, it was decided to adapt the new method published by Poethko et al., Two-Step Methodology for High-Yield Routine Radiohalogenation of Peptides: (18)F-labeled RGD and Octreotide Analogs. J. Nucl. Med. 2004 May; 45(5):892-902, because the yield of precursor (4-[¹⁸F] fluorobenzoaldehyde, [¹⁸F-]FBA) was much greater than with the earlier method (80-90% vs. 2%). Additionally, Poethko et al. reported no problem with dissociation of ¹⁸F from the ¹⁸F-labeled peptide. Thus ¹⁸F-FBA was prepared following Poethko's method (FIG. 8). In addition, a ¹⁹F linker was again used to optimize the cross-linking conditions, and the process was elucidated with MALDI-TOF Mass Spectrometer. However, the Aoa-TN14003 was unstable and it did not react with [¹⁸F]-FBA once it was stored longer than a few weeks, even at −80° C. Due to this, another alternative method was developed as described below.

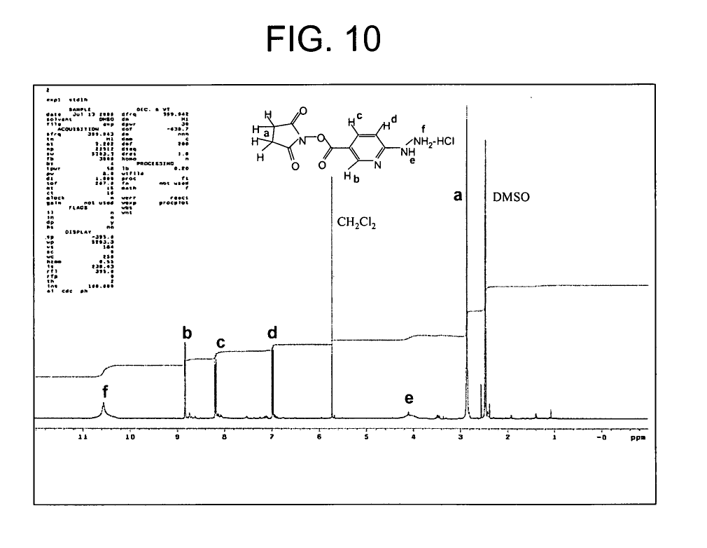

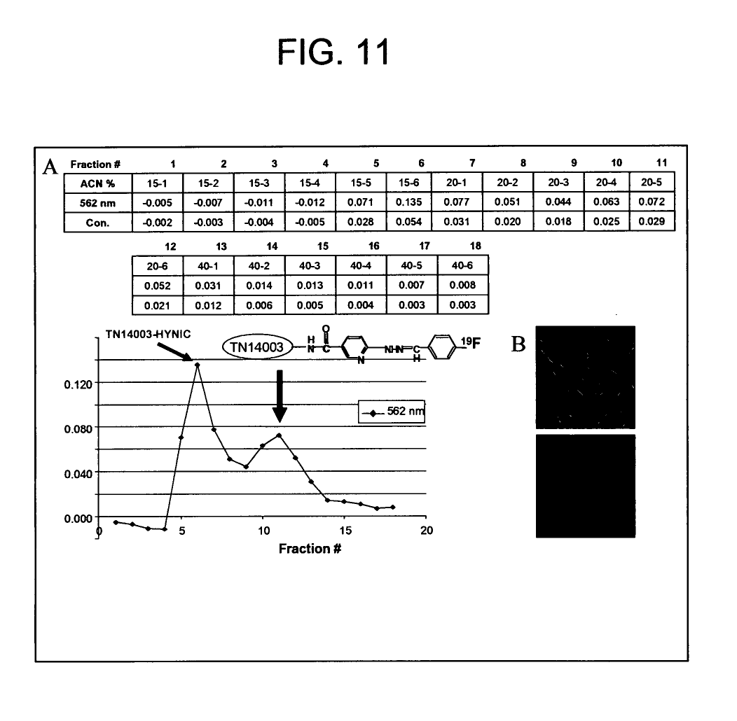

Thus, a method reported by Chang et al. was adapted that includes the preparation of [¹⁸F]-fluorobenzaldehyde ([¹⁸F]-FBA) and the successive conjugation with hydrazinonicotinic acid-human serum albumin conjugate (HYNIC-HSA) via hydrazone formation. Chang, et al., Preparation of 18F-Human Serum Albumin: A Simple and Efficient Protein Labeling Method with 18F using a Hydrazone-formation Method. Bioconjug Chem. 2005 Sep.-Oct.; 16(5): 1329-33. This method was developed to label a large protein, such as human serum albumin and, thus, needed modification for labeling a small peptide (FIG. 9). To label a 14-mer peptide with fluorine, the N-hydroxysuccinimide ester of hydrazinonicotinic acid (NHS-HYNIC) was first prepared according to previously reported methods (37, 71). Briefly, N,N-dimethylalininobenzaldehyde (NHS-HYNIC; 100 mg, 0.44 mmol) was added to an evacuated and argon-purged 25 ml sidearm flask with a stirring bar. Methylene chloride (7 ml) was added to the flask while stirring the solution, which produced a clear slightly greenish-yellow solution. Methyl trifluoromethanesulfonate (53.75/L, 0.475 mmol) was subsequently added, which resulted in an immediate color change to intense yellow. After stirring overnight, the crude product was obtained by the addition of diethyl ether as a yellowish powder (0.14 g). Recrystallization from methylene chloride/diethyl ether produced a fine light yellow crystalline powder. NHS-HYNIC was confirmed by using NMR (FIG. 10). The TN14003 was conjugated with HYNIC by mixing NHS-HYNIC and TN14003. 20 μl of 67.5 mM NHS-HYNIC solution in N,N-dimethylformamide (DMF) was added to 0.3 mL of 3 mM TN14003 solution in 0.1 M sodium bicarbonate (pH 8.3). The molar ratio of HYNIC to TN14003 was 1.5:1. After incubation overnight at 4° C., the TN14003-HYNIC (1:1) was purified by HPLC. A Microsorb C18 (4.6×250 mm) column was used, detecting at 235 nm, and eluting at 25° C. using a linear gradient of acetonitrile in 0.1% TFA for 40 minutes. The purified complex was confirmed by Mass Spectroscopy. TN14003-HYNIC (1:1) was lyophilized overnight, alliquoted, and kept at −20° C. until use (stable for months). The next step was to test the conjugation of TN14003-HYNIC with non-radioactive [¹⁹F]-fluorobenzaldehyde ([¹⁹F]-FBA) to further develop a high yield method of labeling. Fluorine-19 is the same as fluorine-18 without radioactivity. Thus, fluorine-19 was used for this task. [¹⁹F]-FBA was commercially available from Sigma Chemicals. The TN14003-HYNIC and [¹⁹F]-FBA (1:1) were mixed, and the solution was incubated for 30 min at 50° C. The labeled ¹⁹F-TN14003 was then purified with a C18 Sep-Pak using a gradient of acetonitrile (reverse phase-HPLC). The [¹⁹F] was removed at the early phase (0.1% TFA in water) and TN14003-HYNIC eliminated at the medium phase (12% acetonitrile, 0.1% TFA in water) (FIG. 11A). The ¹⁹F-TN14003 was collected at 20% acetonitrile and 0.1% TFA in water. The final product was confirmed by Mass Spectroscopy. The reaction efficacy of TN14003-HYNIC and [¹⁹F]-FBA was almost 50%, much greater than that in the original Garg's method.

Using biotin-labeled CXCR4 antagonist along with streptavidin-conjugated rhodamine, the binding efficiency of the fluorine 19-labeled CXCR4 antagonist to CXCR4 receptor on tumor cells was determined and compared to that of the unlabeled CXCR4 antagonist in immunofluorescence as was done for drug screening, described above. Cells incubated with only streptavidin-conjugated rhodamine (negative control) were compared to cells incubated with both the biotin-labeled CXCR4 antagonist and streptavidin-conjugated rhodamine (positive control). When equal amounts of unlabeled CXCR4 antagonist were added into the biotin-labeled antagonist mixture, the red fluorescence decreased due to the competition between biotin-labeled antagonist and unlabeled antagonist. The same amount of red fluorescence decrease was seen when fluorine 19-labeled antagonist was added to the biotin-labeled antagonist mixture (FIG. 11B). This demonstrates that the binding efficiency of the fluorine-labeled antagonist is the same as that of unlabeled (original) antagonist. Therefore, fluorine labeling did not affect binding efficiency of the CXCR4 antagonist to the CXCR4 protein on the cell surface. However, when the reaction was carried out with fluorine-18, the actual molarity of FBA in 10 mCi [¹⁸F]-FBA was only in the subnano molar range. Thus, there will be much more TN14003-HYNIC than [¹⁸F]-FBA. Therefore, more TN14003-HYNIC was used than [¹⁹F]-FBA.

Once the reaction conditions are properly optimized using fluorine-1 g, the conjugation using [¹⁸F]-FBA produced with fluorine-18 from on-site cyclotron was tested. The process to increase the conjugation yield and purify the 18F-labeled peptide was further optimized to allow separation of the ¹⁸F-labeled peptide from unlabeled peptide as well as [¹⁸F]fluorobenzoaldehyde. To increase the yield of conjugation, the ratio of TN14003-HYNIC was varied over [¹⁸F]fluorobenzoaldehyde to achieve almost 90% reactivity.

The purification of the ¹⁸F-TN14003 was carried out as it was described before for [¹⁹F] compound. Briefly, the ¹⁸F-TN1400 was separated from unreacted TN14003-HYNIC and [¹⁸F]FBA by reverse phase HPLC using a C18 Sep-Pak with a gradient of acetonitrile. The [¹⁸F] was removed at the early phase (0.1% TFA in water) and unreacted TN14003-HYNIC was eliminated at the second phase (12% acetonitrile, 0.1% TFA in water). The fraction with the ¹⁸F-TN14003 was collected at the third phase (20% acetonitrile, 0.1% TFA in water). Then [¹⁸F]FBS gets eluted at the last phase with 40% acetonitrile in 0.1% TFA in water. This process is illustrated schematically in FIG. 12.

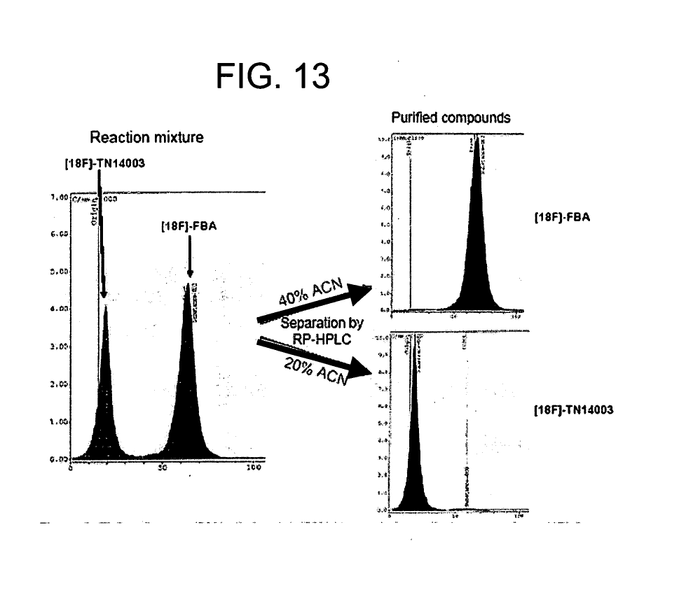

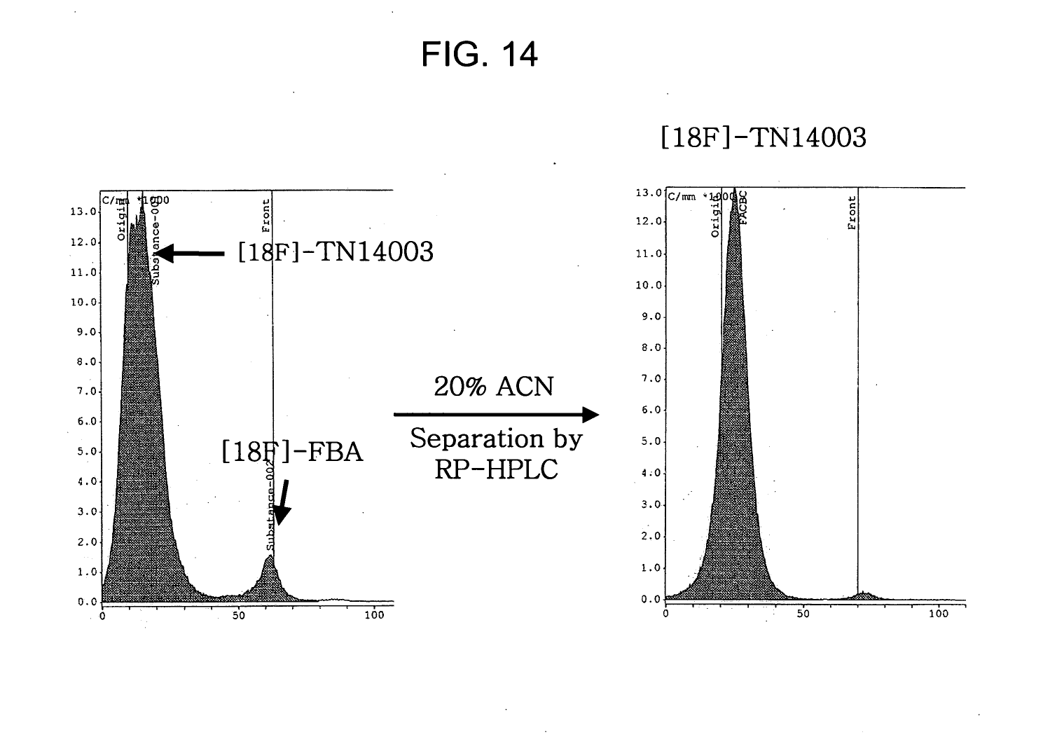

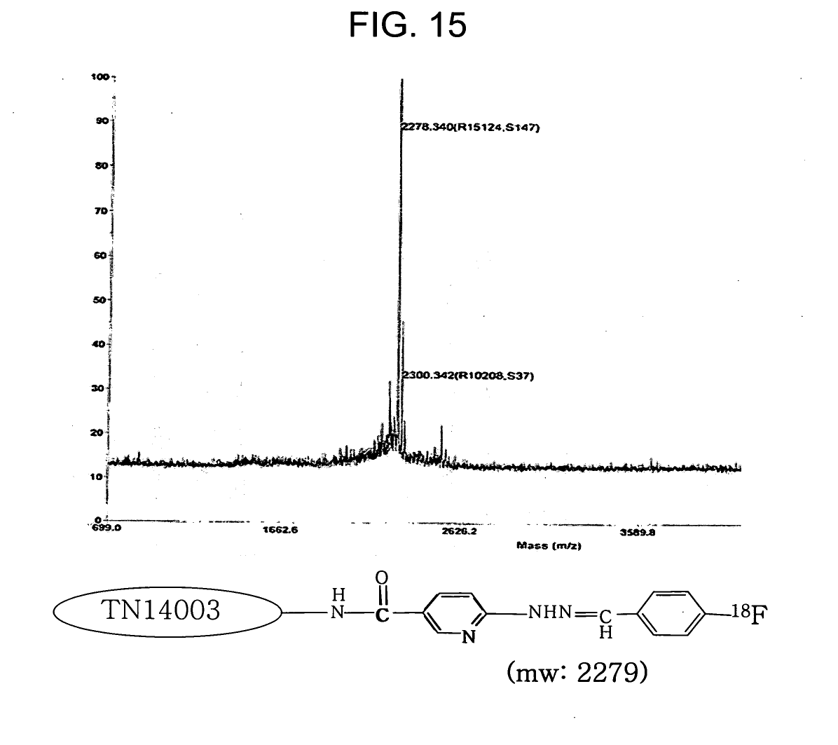

FIG. 13 shows the thin layer chromatography (TLC) radiogram of mixture of [18F]fluorobenzoaldehyde and HYNIC-conjugated CXCR4 antagonist. Following the reverse phase-HPLC, ¹⁸F-conjugated CXCR4 antagonist was eluted at 20% of acetonitrile whereas [¹⁸F]fluorobenzoaldehyde was eluted at 40% of acetonitrile. FIG. 14 further demonstrates the much better yield of improved conjugation, showing almost 90% reactivity, when the ratios of TN14003-HYNIC to [¹⁸F]FBA were varied, as described above. The purified fluorinated CXCR4 antagonist was confirmed by mass spectroscopy. The calculated mess of the final product was 2279 (FIG. 15), which was the same as the experimental data. This process allows for quick and efficient separation of the labeled product from the label and unlabeled product, which is critical when working with radioactive materials.

In summary, because CXCR4 is a critical factor for SCCHN metastasis, PET biomarkers for CXCR4 will be highly useful for discriminating tumors with the greatest risk of metastatic spread and for early detection of metastasis. Routine application of ¹⁸F-labeled peptides for quantitative in vivo receptor imaging using PET is limited by the lack of appropriate radiofluorination methods for routine large-scale synthesis of ¹⁸F-labeled peptides. A classical method developed by Garg et al. two decades ago was to label monoclonal antibody fragments with ¹⁸F using N-succinimidyl 4-[¹⁸F](fluoromethyl) benzoate (SFB). However, this method proved unsuitable for peptide labeling, thus, in the above examples a breakthrough method by Poethko et al. was adapted. This proved to be an excellent method if a fresh batch of aminooxy-functionalized peptide can be synthesized immediately before the ¹⁸F labeling steps. The CXCR4 antagonist of the present disclosure is a 14-mer synthetic peptide that takes four weeks of preparation due to multiple purification steps. Since an aminooxy-functionalized CXCR4 antagonist was found to be extremely unstable; thus, a new method reported by Chang et al was modified for labeling the CXCR4 peptide antagonist. This method includes a novel and simple method for the preparation of [¹⁸F]-fluorobenzaldehyde ([¹⁸F]-FBA) and successive conjugation with hydrazinonicotinic acid-human serum albumin conjugate (HYNIC-HSA) via hydrazine formation. This method was further modified in the present example by adding steps to further increase the yield as well as the purity of ¹⁸F-labeled CXCR4 antagonist from unlabeled CXCR4 antagonist, which is crucial for receptor imaging. It is believed that the methods described above can also be applied to label other peptides for imaging applications.

We claim:

1. An imaging composition comprising:
a CXCR4 peptide antagonist, wherein the CXCR4 peptide antagonist is not an antibody or fragment thereof; and
a radioisotope coupled to the CXCR4 peptide antagonist, wherein the radioisotope is detectable by a PET scanner.

2. The composition of claim 1, wherein the radioisotope is selected from ¹¹C, ¹⁸F, ⁷⁶Br, ¹²³I, ¹²⁴I, and ¹³¹I.

3. The composition of claim 1, further comprising a pharmaceutically acceptable carrier.

4. The composition of claim 1, wherein the CXCR4 peptide antagonist is TN14003 or a derivative thereof.

5. The composition of claim 1, wherein the CXCR4 peptide antagonist interferes with ligand binding to a CXCR4 receptor or homologue thereof.

6. The composition of claim 1, wherein the CXCR4 peptide antagonist specifically binds to CXCR4 receptors and thereby prevents SDF-1 binding.

7. The composition of claim 1, wherein the radioisotope is ¹⁸F.

8. The composition of claim 7, wherein the ¹⁸F is coupled to the CXCR4 peptide antagonist via a linker comprising hydrazinonicotinic acid (HYNIC).

9. An imaging composition comprising:
an ¹⁸F labeled CXCR4 peptide antagonist, wherein the CXCR4 peptide antagonist is TN14003 or a derivative thereof; and
a pharmaceutically acceptable carrier.

10. A method of imaging comprising:
providing an imaging probe comprising a CXCR4 peptide antagonist coupled to a radioisotope, wherein the CXCR4 peptide antagonist is not an antibody or fragment thereof.
contacting a specimen to be imaged with a detectably effective amount of the imaging probe; and
making a radiographic image.

11. The method of claim 10, wherein the specimen is selected from: a cell, tissue, and a host.

12. The method of claim 11, wherein the host is a mammal.

13. The method of claim 11, wherein the tissue comprises tumor tissue.

14. The method of claim 10, wherein making the radiographic image comprises using an imaging apparatus and wherein the imaging apparatus is selected from: a gamma camera, a PET apparatus, and a SPECT apparatus.

15. The method of claim 14, wherein the imaging apparatus is a PET apparatus and the radioisotope is selected from ¹¹C, ¹⁸F, ⁷⁶Br, ¹²³I, ¹²⁴I, and ¹³¹I.

16. The method of claim 15, wherein the radioisotope is ¹⁸F.

17. The method of claim 10, wherein the CXCR4 peptide antagonist is TN14003 or a derivative thereof.

18. The method of claim 17, wherein the imaging probe comprises ¹⁸F-TN14003.

19. The method of claim 10, wherein the imaging comprises imaging expression of CXCR4 receptors, wherein the expression of CXCR4 receptors is associated with one or more of: inflammation, cancer, a tumor, angiogenesis, and metastasis.

20. The method of claim 10, wherein the imaging comprises detecting cancer in the specimen.

21. The method of claim 10, wherein the imaging comprises detecting or predicting metastasis of a tumor in the specimen.

22. A method of imaging a condition associated with expression of CXCR4 receptors in a host comprising:
administering to the host a detectably effective amount of a composition comprising a radiolabeled CXCR4 peptide antagonist, wherein the CXCR4 peptide antagonist is not an antibody or fragment thereof; and
creating a radiographic image of the location and distribution of the a radiolabeled CXCR4 peptide antagonist in the host with an imaging apparatus,
wherein the radiolabeled CXCR4 peptide antagonist binds to CXCR4 receptors, and wherein the intensity of uptake of radiolabeled CXCR4 peptide antagonist is related to the expression level of CXCR4 receptors in the host, and wherein the expression level of CXCR4 receptors is associated with one or more disorders.

23. The method of claim 22, wherein the disorder associated with expression of CXCR4 receptors is selected from one or more of: inflammation, cancer, a tumor, angiogenesis, and metastasis.

24. The method of claim 22, wherein the imaging apparatus is selected from: a gamma camera, a PET apparatus, and a SPECT apparatus.

25. The method of claim 22, wherein imaging a condition associated with expression of CXCR4 receptors comprises diagnosing the condition or monitoring the condition.

26. The method of claim 22, wherein the imaging probe comprises ¹⁸F-TN14003.

27. A method of predicting metastasis comprising:
contacting a specimen comprising tumor cells with a detectably effective amount of a composition comprising ¹⁸F-TN14003; and
creating a radiographic image of the location and distribution of the ¹⁸F-TN14003 in the tumor cells with an imaging apparatus,
wherein the ¹⁸F-TN14003 binds to CXCR4 receptors, and
wherein the intensity of uptake of ¹⁸F-TN14003 by the tumor cells is related to the metastatic potential of the tumor cells.

28. The method of claim 27, wherein contacting a specimen comprising tumor cells with a detectably effective amount of a composition comprising ¹⁸F-TN14003 comprises administering to a host with cancer a detectably effective amount of a composition comprising ¹⁸F-TN14003.

29. The method of claim 27, wherein the specimen comprising tumor cells comprises a tissue sample obtained from a tumor biopsy from a host with cancer.

30. A method of determining an effect of a drug comprising:
administering an amount of the drug to a host with cancer;
administering a detectably effective amount of a composition comprising ¹⁸F-TN14003 to a host;
creating a radiographic image of the location and distribution of the ¹⁸F-TN14003 in the host with an imaging apparatus, and
determining an amount of ¹⁸F-TN14003 taken up by host cancer cells, wherein the amount of uptake of ¹⁸F-TN14003 is related to the effect of the drug for treating cancer in the host.

31. A method of synthesizing ¹⁸F-TN14003 comprising the steps of:
providing N-hydroxysuccinimide ester of hydrazinonicotinic acid (NHS-HYNIC);
mixing the NHS-HYNIC with TN14003 to form TN14003-HYNIC;
mixing the TN14003-HYNIC with [¹⁸F]-fluorobenzaldehyde ([¹⁸F]FBA) to form ¹⁸F-TN14003-HYNIC (18F-TN14003); and
separating ¹⁸F-TN14003 from unreacted TN14003-HYNIC and [¹⁸F]FBA to obtain substantially pure ¹⁸F-TN14003.

32. The method of claim 31, wherein separating ¹⁸F-TN14003 from unreacted TN14003-HYNIC and [¹⁸F]FBA comprises using reverse phase HPLC.

33. The method of claim 32, wherein the reverse phase HPLC comprises using a C18 Sep-Pak with a gradient of acetonitrile, wherein the [¹⁸F] is eluted at about 0.1% TFA in water;
the unreacted TN14003-HYNIC is eluted at about 12% acetonitrile, 0.1% TFA in water;
the ¹⁸F-TN14003 is eluted at about 20% acetonitrile, 0.1% TFA in water; and
the [¹⁸F]FBS is eluted at about 40% acetonitrile in 0.1% TFA in water.

34. The method of claim 31 further comprising: increasing the yield of ¹⁸F-TN14003 by varying the ratio of TN14003-HYNIC to [¹⁸F]FBA.
